(19) United States
(12) Patent Application Publication
(10) Pub. No.: US 2025/0000836 A1
(43) Pub. Date: Jan. 2, 2025

(54) PARTICLE DELIVERY OF THYROID HORMONE RECEPTOR AGONISTS AND ANTAGONISTS

(71) Applicants: CYTA THERAPEUTICS, INC., Lowell, MA (US); UNIVERSITY OF MASSACHUSETTS, Boston, MA (US)

(72) Inventors: Stephen W. FARACI, Arlington, MA (US); Roman HERRERA, Philadelphia, PA (US); Bernadette C. FENDROCK, Gloucester, MA (US); Sankaran THAYUMANAVAN, Amherst, MA (US); Hang XIAO, Amherst, MA (US); Ruiling WU, Boston, MA (US)

(21) Appl. No.: 18/580,737

(22) PCT Filed: Jul. 21, 2022
(86) PCT No.: PCT/US2022/037814

§ 371 Date: Jan. 19, 2024

Related U.S. Application Data
(60) Provisional application No. 63/224,134, filed on Jul. 21, 2021.

(51) Int. Cl. A61K 31/225; A61K 9/06; A61K 9/51; A61P 5/16

(52) U.S. Cl. CPC A61K 31/225; A61K 9/06; A61K 9/5146; A61K 9/5169; A61P 5/16

(57) ABSTRACT

An aspect of the present invention provides tissue-selective delivery of thyroid hormone receptor (TR)β agonists or antagonists using particle delivery systems. Specifically, aspects of the invention provide pharmaceutical compositions comprising effective amounts of a TRβ agonist or antagonist encapsulated in a particle carrier, as well as methods for using the same to treat various medical diseases and conditions. In various embodiments, the compositions mediate selective TRβ activation in a tissue selective manner.

### TECHNICAL FIELD OF THE INVENTION

The invention generally relates to nanotechnology and drug delivery. More particularly, the invention relates to compositions and methods for encapsulation and tissue-selective delivery of thyroid hormone receptor-β agonists and antagonists for treatment of certain diseases and conditions.

### BACKGROUND OF THE INVENTION

Thyroid hormones (THs), 3,3′,5-triiodo-L-thyronine (T3) and 3,5,3′,5′-tetraiodo-1-thyronine (thyroxine or T4), impact processes and pathways mediating carbohydrate, lipid, protein, and mineral metabolism in almost all tissues. There is particular interest in the potential for administration of thyroid hormone receptor agonists to affect lipid metabolism, for example to increase metabolic rate, weight loss, lipolysis, and lowering of serum cholesterol levels. However, these therapeutically desirable effects can be associated with a thyrotoxic state, which includes induction of tachycardia, arrhythmia, muscle catabolism, reduced bone mineralization, alteration of central nervous system (CNS) development, and mood disorders. The effects of THs, as well as thyroid receptor agonists and antagonists, are mediated by nuclear receptors: thyroid hormone receptor-α (Rα) and thyroid hormone receptor-β (TRβ). Thyroid receptors are quite heterogeneous among different tissues; for example, TRα is the dominant receptor in the brain and skeletal system and mediates most of the synergism between T3 and the sympathetic signaling pathway in the heart, while TRβ is the most abundant TH isoform in the liver where it mediates most of the T3 effects on lipid metabolism and regulation of metabolic rate.

There is a need for harnessing the beneficial physiological effects of thyroid hormone receptor agonists and antagonists (e.g., in select tissues such as the liver), uncoupled from undesired responses (e.g., in other tissues such as heart or CNS). In the various aspects and embodiments, the invention meets these objectives.

### SUMMARY OF THE INVENTION

An aspect of the present invention provides tissue-selective delivery of thyroid hormone receptor (TR)β agonists or antagonists using particle delivery systems. Specifically, aspects of the invention provide novel nanogels and pharmaceutical compositions comprising effective amounts of a TRβ agonist or antagonist encapsulated in a particle carrier, as well as methods for using the same to treat various medical conditions. In various embodiments, the compositions mediate selective TRβ activation in a tissue selective manner.

In certain aspects and embodiments, the invention provides a pharmaceutical composition comprising an effective amount of a TRβ agonist or antagonist encapsulated in a pharmaceutically acceptable particle carrier, wherein the TRβ agonist or TRβ antagonist is released upon degradation of the particle carrier. For example, in various embodiments, the particle carrier does not degrade in the circulation, but degradation is triggered upon internalization by target cells or tissues.

In various embodiments, the particle carrier accumulates in or is targeted to a particular organ or tissue, and the particle carrier may comprise a targeting agent. In some embodiments, the particle carrier is targeted to the liver. In some embodiments, the targeting agent is an anionic functionality that targets organic anion-transporting polypeptide (OATP) group of receptors. For example, the anionic functionality may be a carboxylate, which can include a disulfide functionality to support degradation of the particle carrier.

TRβ agonists include but are not limited to natural thyroid hormones, as well as derivatives of thyroid hormones that bind to TRβ and affect cell or tissue functions. In some embodiments, the composition comprises the TRα agonist is axitirome (CGS26214).

In some embodiments, the particle carrier has an average diameter in the range of about 10 nm to about 200 nm, or in the range of about 20 nm to about 100 nm. In various embodiments, the carrier is less than 100 nm in average diameter. In some embodiments, the particle carrier has a zeta potential in the range of about −5 mV to about −40 mV, or in the range of about −10 mV to about −30 mV.

The TRβ agonist or antagonist may be non-covalently incorporated into the particle carrier. For example, the TRβ agonist or antagonist may be non-covalently incorporated into a crosslinked or non-crosslinked network of polymer molecules. In other embodiments, the TRβ agonist or TRβ antagonist is covalently linked to the particle carrier and is released upon degradation of the carrier. In some embodiments, the TRβ agonist or TRβ antagonist is incorporated in the particle carrier non-covalently, where the particle carrier is polymeric and comprises a crosslinked interior, where degradation of the carrier is triggered by an increased concentration of a biochemical reductant. For example, in some embodiments the particle carrier degrades in the presence of intracellular concentrations of glutathione (GSH), but the carrier does not substantially degrade in plasma (i.e., will not substantially degrade in the circulation).

In some embodiments, the particle carrier is formed by self-assembly in an aqueous environment. In some embodiments, the carrier comprises an oligoethylene glycol (OEG, used herein interchangeably with polyethylene glycol or PEG) hydrophilic shell and a hydrophobic interior comprising disulfide-crosslinked branch groups, allowing the carrier to degrade in the presence of intracellular concentrations of GSH. In various embodiments, the hydrophobic interior comprises hydrophobic branch groups (having a hydrophobic moiety) to drive particle assembly and allow crosslinking of the interior For example, the hydrophobic branch groups may comprise pyridyldisulfide (PDS) moieties.

The amphiphilic nature of the particle carrier and hydrophobic environment provide the opportunity for hydrophobic guest molecules (such as the TRβ agonist or antagonist), to be sequestered within the nano-assemblies prior to crosslinking. Further, since the particle carriers may be based on disulfide crosslinkers that can be cleaved by thiol-disulfide exchange reactions, the nanogels also have a pathway to release the stably encapsulated guest molecules.

The pharmaceutical composition can be targeted to the liver selectively over other tissues by incorporating an anionic functionality into the particle carrier, which targets OATP group of receptors. Alternatively, or in addition, the pharmaceutical composition may comprise other targeting schemes to direct the particle carrier to target tissues or cells. Such targeting will improve the efficiency and effectiveness of the guest molecule, such as a TRβ agonist, as the local concentration of the guest molecule is elevated. In some embodiments, the targeting agent may be a tissue selective targeting agent, or may be selective for certain cells, such as but not limited to hepatocytes.

In exemplary embodiments, the invention provides a pharmaceutical composition comprising a particle carrier non-covalently encapsulating an effective amount of a TRβ agonist, such as axitirome. The particle carrier comprises an anionic functionality that targets OATP group of receptors (which in some embodiments is a carboxylate functionality), and the particle carrier comprises a disulfide-crosslinked polymeric interior that is not substantially degraded in normal blood plasma and is substantially degraded in the presence of intracellular concentrations of GSH. In such embodiments, the particle carrier has an average diameter in the range of about 10 nm to about 200 nm, or in the range of about 20 nm to about 100 nm, and has a zeta potential in the range of about −5 mV to about −40 mV. The particle carrier is formed by self-assembly in an aqueous environment. For example, the particle carrier is formed in the presence of the TRβ agonist axitirome and an amphiphilic copolymer. The amphiphilic copolymer comprises hydrophilic OEG branch groups and disulfide-linked hydrophobic branch groups (e.g., PDS moieties) to drive micellar assembly and agonist encapsulation, followed by cross-linking of hydrophobic branch groups through disulfide exchange reactions. In various embodiments, the particle carrier has a crosslinking density of at least about 10%, or at least about 20%, such from about 10% to about 70%, relative to the number of structural units in the polymer. In some embodiments, the density of the anionic ligand is about 35% to about 45% (e.g., about 40%) with respect to total structural units in the polymer.

The TRβ agonist or antagonist, is released upon partial or complete degradation or de-crosslinking of polymer molecules at or near the biological site. For example, after transport of the particle carrier to the target tissue or cells, the carrier may be degraded or de-crosslinked, thereby releasing the active agent. In one embodiment, the degradation is triggered by an endosomal or intracellular environment upon cell internalization. For example, the degradation may be caused by breaking the disulfide bonds in the particle carrier in a reducing environment. In some embodiments, the active agent is not substantially released at concentrations of reducing agent characteristic of blood plasma, so that active agent is only released after cell internalization.

In other aspects, the invention provides a method for treating a disease or condition, comprising administering an effective amount of the pharmaceutical composition described herein to a patient in need thereof. In some embodiments, the patient has non-alcoholic fatty liver disease (NAFLD) (e.g., NASH), and the particle carrier encapsulates a TRβ agonist, such as but not limited to axitirome. In some embodiments, the patient to be treated may have type 1 or type 2 diabetes, or metabolic syndrome. For example, the patient to be treated may be obese or overweight. In some embodiments, the patient may be hypercholesterolemic or hyperlipidemic. In such embodiments, the TRβ agonist stimulates liver metabolism. The method can result in one or more of: lowering of total serum cholesterol, lowering LDL cholesterol, lowering of serum triglycerides, lowering of serum lipoprotein A, decrease in hepatic fat, increase in lipolysis, increase in hepatocyte proliferation, and weight loss.

In certain aspects and embodiments, the invention provides a nanogel comprising a crosslinked copolymer and a thyroid hormone receptor-β (TRβ) agonist or a TRβ antagonist encapsulated in the crosslinked polymer.

In certain embodiments, the crosslinked copolymer composes the structural units of the following structural formula (although it is understood that the order of monomers in the co-polymer is essentially random):

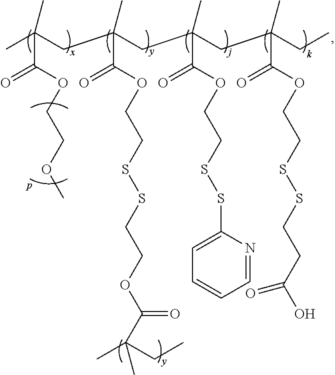

wherein j is percentage of (x+y+j+k) in the range from 0% to about 70% (e.g., 10% to 50%, or 10% to 40%, or 10% to 30%, or 10% to 20%, or 20 to 40%), x and k are independently in the range from 1% to about 50% (e.g., independently 10% to 50%, or 20% to 50%, or 30% to 50%).

Other aspects and embodiments of the invention will be apparent from the following detailed description.

### BRIEF DESCRIPTION OF THE DRAWINGS

FIG. 1. Synthesis of CGS26214-encapsulated nanogel with anionic ligand modification.

FIG. 2. Characterization of CGS26214-encapsulated nanogel with anionic ligand modification. Nanogel size and zeta potential was determined by DLS.

FIGS. 3A-3D. Shows body weight (A), liver weight (B), heart weight (C), and epididymal fat pad weight (D) of the different treatment groups for the 12-week study.

FIGS. 4A-4D. Shows body weight (A), liver weight (B), heart weight (C), and epididymal fat pad weight (D) of the different treatment groups for the 24-week study.

FIG. 5. Representative images (body size) of mice from different treatment groups.

FIG. 6. Representative images of livers from mice from different treatment groups.

FIG. 7. Liver histology of selected mice from different treatment groups using hematoxylin-eosin staining, 20× magnification, scale bar 100 μm. Mice from treatment groups HFD, CGS-2 and BNG show macrovesicular and microvesicular steatosis, inflammation, and ballooning degeneration while mice from treatment groups CD and CNG-2 show no signs of macrovesicular and microvesicular steatosis, inflammation, and ballooning.

FIG. 8. Liver histology of selected mice from different treatment groups using Masson's trichrome staining, 20× magnification, scale bar 100 μm. Mice from treatment groups HFD, CGS-2 and BNG show signs of fibrosis and collagenous tissue fiber formation while mice from treatment groups CD and CNG-2 show no signs of fibrosis or collagen deposition.

FIG. 9. Regulation of gene transcription activity in the liver (CYP7A1 expression, SREBP-1c expression, and LDLR expression) of all treatment groups at 24 weeks. Data are shown as means±SE of 8-10 animals/group.

FIGS. 10A and 10B. Serum thyroid hormone (T4) and thyroid stimulating hormone (TSH) levels of Diet Induced Obese (DIO) mice treated with CGS26214 (drug) and CGS26214-encapsulated nanogels with anionic group-modified backbone (CYTA-001) at 12 weeks (A) and 24 weeks (B).

FIG. 11. Homeostatic model assessment for insulin resistance (HOMA-IR) levels in DIO mice on a 24-week high fat diet (HFD) and after 5-week dosing regimen of CGS26214 (drug) and CGS26214-encapsulated nanogels with anionic group-modified backbone (CYTA-001).

FIG. 12. In vivo fluorescence microscopy imaging of the biodistribution of subcutaneously-delivered Cy3β-tagged (fluorescent dye, ThermoFisher) nanogel with anionic group-modified backbone. Data are shown as pairs of in vivo fluorescence imaging of the frontal plane (left) and ex vivo fluorescence imaging of individual organs, brain liver, heart, lung, and kidney (right) at 1 hour (h), 4 h, 8 h, 12 h, and 24 h post injection.

### DETAILED DESCRIPTION OF THE INVENTION

An aspect of the present invention provides tissue-selective delivery of thyroid hormone receptor (TR)β agonists or antagonists using particle delivery systems. Specifically, aspects of the invention provide pharmaceutical compositions comprising effective amounts of a TRβ agonist or antagonist encapsulated in a particle carrier, as well as methods for using the same. In various embodiments, the compositions mediate selective TRβ activation in a tissue selective manner.

Selective activation of TRβ is useful for ameliorating a variety of metabolic disorders, including metabolic syndrome, type 2 diabetes, hypercholesterolemia, hyperlipidemia, non-alcoholic steatohepatitis, liver fibrosis and obesity, among others. In particular, nonalcoholic fatty liver disease (NAFLD) represents a spectrum of hepatic disorders that range from excess lipid storage in the liver (hepatosteatosis) to progressive nonalcoholic steatohepatitis (NASH), which can lead to cirrhosis and hepatocellular cancer. NAFLD has recently become a pandemic that affects approximately 25% of adults worldwide, with its prevalence estimated to be 60% to 80% in patients with type 2 diabetes mellitus (DM) and obesity. Currently, there are no U.S. Food and Drug Administration approved pharmacological therapies for NASH, and liver transplantation is the only treatment for end stage NAFLD. Thyroid hormones stimulate fatty acid n-oxidation and oxidative phosphorylation in the liver and clinical studies have shown an inverse relationship between serum thyroid hormone levels and NAFLD (i.e., patients with hypothyroidism have increased risk for NAFLD). TRβ acts via a pleiotropic mechanism, in which multiple metabolic enzymes and pathways are impacted upon receptor activation, including adipose triglyceride lipase, carnitine palmitoyl-transferase 1α, mitochondrial autophagy and biogenesis, and cholesterol 7α hydroxylase.

Selective inhibition of TRβ is useful for ameliorating a thyrotoxic state (“thyrotoxicosis”). Thyrotoxicosis is a clinical state of inappropriately high levels of circulating thyroid hormones (T3 and/or T4) in the body. Medical conditions sometimes associated with thyrotoxicosis include Grave's disease, toxic multinodular goiter, toxic adenoma, TSH-producing adenoma or pituitary adenoma, HCG-mediated hyperthyroidism, thyroiditis, drug-induced increased secretion of thyroid hormone (e.g., induced by amiodarone or iodinated contrast), factitious hyperthyroidism, and excessive replacement therapy (e.g., with levothyroxine).

In certain aspects and embodiments, the invention provides a pharmaceutical composition comprising an effective amount of a thyroid hormone receptor-β (TRβ) agonist or a TRβ antagonist encapsulated in a pharmaceutically acceptable particle carrier, wherein the TRβ agonist or TRβ antagonist is released upon degradation of the particle carrier. For example, in various embodiments, the particle carrier does not degrade in the circulation, but degradation is triggered upon internalization by target cells or tissues.

In various embodiments, the particle carrier accumulates in or is targeted to an organ or tissue, and the particle carrier may comprise a targeting agent. For example, in various embodiments, the particle carrier can accumulate or be targeted to an organ, tissue, or cell selected from liver, kidney, lung, heart, nerves, macrophages, hematopoietic stem cells, hepatic stellate cells, vasculature, brain, vagina, uterus, stomach, intestine (small and large intestine), or muscles of specific organs. In some embodiments, the particle carrier is targeted to the liver.

In some embodiments, the targeting agent is an anionic functionality that targets OATP group of receptors, which are membrane transport proteins that mediate the transport of mainly organic anions across the cell membrane. OATPs are present in the lipid bilayer of the cell membrane. OATPs carry bile acids as well as bilirubin and numerous hormones such as thyroid and steroid hormones across the basolateral membrane in hepatocytes. As well as expression in the liver, various OATPs are expressed in other tissues on basolateral and apical membranes. In some embodiments, the anionic functionality is a carboxylate, including a C1 to C12 or C2 to C8 (e.g., C2, C3, C4, C5, or C6) carboxylate, which can include a disulfide functionality to support degradation of the particle as described herein. An exemplary anionic functionality can be created by incorporation of mercaptocarboxylic acid compound (e.g., mercaptopropionic acid) into particles. In some embodiments, the carrier comprises a propionate targeting moiety conjugated to the particle via a disulfide bond, which mediates targeting to the liver.

In various embodiments, the density of the anionic ligand is about 10% to about 60% with respect to total structural units in the polymer, or about 20% to about 60%, or about 30% to about 50% with respect to total structural units in the polymer. In some embodiments, the density of the anionic ligand is about 35% to about 45% (e.g., about 40%) with respect to total structural units in the polymer.

It will be understood by one of skill in the art that the term “structural unit” means the monomer units that form the resulting co-polymer, and result in x, y, j, and k in the structural formula provided herein.

In various aspects and embodiments, the present invention allows for the medical potential of various TRβ agonists or TRβ antagonists to be realized, and in particular those that have medically important tissue-specific or cell-specific biological effects. TRβ agonists in particular can have biological actions on many different cell types and have a wide variety of biological effects.

TRβ agonists include but are not limited to natural thyroid hormones, as well as derivatives of thyroid hormones that bind to TRβ receptor and affect cell or tissue functions. TRβ antagonists include but are not limited to: any inhibitor of a natural thyroid hormone function by reducing or blocking the signaling cascade of thyroid hormone, and any molecule that reduces or blocks the binding of the thyroid hormone to TRβ, such as but not limited to a thyroid hormone derivative or analog that competitively binds to TRβ and reduces the signaling of TRβ by thyroid hormone binding.

Exemplary TRβ agonists include Triiodothyronine (T3) or its prohormone thyroxine (T4), Sobetirome (GC-1), GC-24, Eprotirome (KB2115), KB141, Resmetirom (MGL-3196), VK2809, Axitirome (CGS26214) or CGS23425, including stereoisomers, as well as any pharmaceutically acceptable salt or prodrug thereof. These and other TRβ agonists, including aryloxyphenyl based thyromimetics and diphenylmethane based thyromimetics, are described in Saponaro F., et al. “Selective Thyroid Hormone Receptor-Beta (TRβ) Agonists: New Perspectives for the Treatment of Metabolic and Neurodegenerative Disorders.” Frontiers in Medicine Vol. 7 Art. 331 (2020). Other TRβ agonists and antagonists are described in Raparti G., “Selective thyroid hormone receptor modulators.” Indian J Endocrinol Metab. 2013 March-April; 17(2): 211-218. In various embodiments, the TRβ agonist or antagonist is hydrophobic.

In some embodiments, the composition comprises a TRβ agonist, and the TRβ agonist is axitirome (CGS26214). Axitirome can be described by the chemical formula: ethyl (+−)-((4-(3-((4-fluorophenyl)hydroxymethyl)-4-hydroxyphenoxy)-3,5-dimethylphenyl)amino)oxoacetate, as well as stereoisomers, pharmaceutically acceptable salts, and prodrugs thereof.

### Axitirome

The pharmaceutical composition comprises a particle carrier, which can be a nanoparticle or microparticle carrier, to deliver the active agent to desired tissues or cells. As used herein, the term “nanoparticle,” refers to a particle having at least one dimension in the range of about 1 nm to about 1000 nm. The term “microparticle” includes particles having at least one dimension in the range of about 1 μm to 100 μm. The term “particle” includes nanoparticles and microparticles. The size of the particle carrier can impact the pharmacodynamics of the composition, including tissue distribution, cell internalization, and size of the payload, for example. In various embodiments, the particle may have a size (i.e., average diameter or length of longest dimension) in the range of about 10 nm to about 5 μm. In various embodiments, the particle carrier may have a size in the range of about 10 nm to about 500 nm, or in the range of about 10 nm to about 250 nm, or in the range of about 10 to 100 nm. In some embodiments, the particle carrier has an average diameter in the range of about 10 nm to about 200 nm, or in the range of about 20 nm to about 100 nm, or in the range of about 25 nm to about 75 nm. In various embodiments, the carrier is less than 100 nm in average diameter. In some embodiments, the particle carrier has a zeta potential in the range of about −5 mV to about −40 mV, or in the range of about −10 mV to about −30 mV (e.g., from about −15 to about −25 mV).

The TRβ agonist or antagonist may be non-covalently incorporated into the particle carrier. For example, the TRβ agonist or antagonist may be non-covalently incorporated into a crosslinked or non-crosslinked network of polymer molecules, which are part of the polymeric carrier. In other embodiments, the TRβ agonist or TRβ antagonist is covalently linked to the nanoparticle or microparticle carrier and is released upon degradation of the carrier. For example, the TRβ agonist or TRβ antagonist may be incorporated in the particle carrier non-covalently, where the particle carrier is polymeric and comprises a crosslinked interior, where degradation of the carrier is triggered by an increased concentration of a biochemical reductant. For example, in some embodiments the particle carrier degrades in the presence of intracellular concentrations of GSH, but the carrier does not substantially degrade in plasma (i.e., will not substantially degrade in the circulation).

In some embodiments, the particle carrier is formed by self-assembly in an aqueous environment. For example, the particles may be formed by self-crosslinking reactions with self-crosslinking polymer as described in US 2014/0112881 A1, which is hereby incorporated by reference in its entirety. In some embodiments, the carrier comprises an OEG hydrophilic shell and a hydrophobic interior comprising disulfide-crosslinked branch groups, allowing the carrier to degrade in the presence of intracellular concentrations of GSH. In these embodiments, the particles may be formed from amphiphilic polymers comprising the hydrophilic OEG branch groups and the hydrophobic branch groups.

The OEG groups include

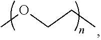

wherein p is an integer from about 5 to about 200 (e.g., from about 5 to about 150, from about 5 to about 100, from about 5 to about 50, from about 10 to about 200, from about 20 to about 200, from about 50 to about 200, from about 100 to about 200, from about 10 to about 30, from about 10 to about 50). In some embodiments, the OEG branch groups have from 5 to 50 ethylene glycol units. OEG units may be used to introduce a charge-neutral hydrophilic functional group, which endows biocompatibility.

In various embodiments, the hydrophobic branch groups comprise a hydrophobic moiety to drive particle assembly and allow crosslinking of the interior. For example, the hydrophobic branch groups may comprise aromatic moieties, such as PDS moieties. The hydrophobic functionality provides a supramolecular amphiphilic nano-assembly in the aqueous phase, which helps avoid the use of any additional surfactant molecules to generate the nanogel. The amphiphilic nature of the particle carrier and hydrophobic environment provide the opportunity for hydrophobic guest molecules (such as the TRβ agonist or antagonist), to be sequestered within these nano-assemblies prior to crosslinking. The PDS functionality is reactive, but specific to thiols, and provides a mild method for disulfide crosslinking to form the nanogel. Furthermore, since the particle carriers may be based on disulfide crosslinkers that can be cleaved by thiol-disulfide exchange reactions, the nanogels also have a pathway to release the stably encapsulated guest molecules. Further, because the particle formation can be conducted with thiol-disulfide exchange or thiol reshuffling reactions, the use of organic solvents and metal containing catalysts or additional reagents can be avoided. In some embodiments, the disulfide exchange reaction may shuffle sulfhydryl groups of dithiothreitol (DTT) into the disulfides of disulfide-linked hydrophobic branch groups.

The OEG branch groups and the hydrophobic branch groups may be present at a ratio of from 1:4 to 4:1 In some embodiments, the OEG branch groups and the hydrophobic branch groups may be present at a ratio of about 1:4, about 1:3, about 1:2, about 1:1, about 2:1, about 3:1 or about 4:1.

The amphiphilic co-polymer may be prepared by reversible addition fragmentation chain transfer (RAFT) polymerization of pyridyl disulfide ethyl methacrylate (PDSEMA) and oligoethylene glycol monomethyl ether methacrylate. The resulting polymer may be purified with precipitation methods. See, for example, US 2014/0112881 A1, which is hereby incorporated by reference in its entirety.

In some embodiments, the crosslinked network of the particle may have a crosslinking density in the range of from 2% to 80%, relative to the total number of disulfide-containing structural units in the polymer. For example, the crosslinked network of may have a crosslinking density from about 2% to about 70%, from about 2% to about 60%, from about 2% to about 50%, from about 2% to about 40%, from about 2% to about 30%, from about 2% to about 20%, from about 2% to about 10%, from about 5% to about 80%, from about 10% to about 80%, from about 20/6 to about 80%, from about 30% to about 80%, from about 40% to about 80%, relative to the total number of disulfide-containing structural units in the polymer. In some embodiments, the crosslinking density is at least about 10%, or at least about 20%, or at least about 30%, relative to the total number of disulfide-containing structural units in the polymer.

Other variations for formulation of particle carriers in accordance with this disclosure may be used, including those described in one or more of US 2014/0112881 A1, US 2015/0202163 A1, US 2015/0209447 A1, and WO 2015/105549 A2, all of which are hereby incorporated by reference in their entireties.

In exemplary embodiments, the particle carrier is formed by self-assembly in an aqueous environment. The particle carrier is formed in the presence of the TRβ agonist and the amphiphilic copolymer. For example, the amphiphilic copolymer comprises hydrophilic OEG branch groups and disulfide-linked hydrophobic branch groups (e.g., pyridyl-containing, or other aromatic-containing, branch groups) to drive micellar assembly and agonist encapsulation, followed by cross-linking of the hydrophobic branch groups through disulfide exchange reactions. In some embodiments, the disulfide exchange reaction shuffles sulfhydryl groups of DTT into the disulfides of disulfide-linked hydrophobic branch groups. In various embodiments, the particle carrier has a crosslinking density from about 10% to 70%, or from about 20% to about 60%, or from about 30% to about 50%, relative to the total number of disulfide-containing structural units in the amphiphilic polymer. In some embodiments, the hydrophobic branch groups comprise PDS moieties. In some embodiments, the OEG branch groups and the hydrophobic branch groups are present at a ratio of from about 1:4 to about 4:1. In some embodiments, the amphiphilic co-polymer is prepared by RAFT polymerization of PDSEMA and oligoethylene glycol monomethyl ether methacrylate. As already described, anionic targeting functions can also be incorporated. In accordance with these embodiments, the TRβ agonist is not substantially released at concentrations of reducing agent found in normal blood plasma. However, the carrier is substantially degraded in the presence of intracellular concentrations of GSH.

Alternatively, the polymeric carrier can comprise other polymeric materials comprising degradable linkages, such as ester linkages, disulfide linkages, amide linkages, anhydride linkages, and a linkage susceptible to enzymatic degradation. For example, the particle carriage may comprise one or more polymers or copolymers selected from cyclodextrin, poly(D,L-lactic acid-co-glycolic acid) (PLGA), poly(caprolactone) (PCL), ethylene vinyl acetate polymer (EVA), poly(lactic acid) (PLA), poly(L-lactic acid) (PLLA), poly(glycolic acid) (PGA), poly(L-lactic acid-co-glycolic acid) (PLLGA), poly(D,L-lactide) (PDLA), poly(L-Lactide) (PLLA), PLGA-b-poly(ethylene glycol)-PLGA (PLGA-bPEG-PLGA), PLLA-bPEG-PLLA, PLGA-PEG, poly(D,L-lactide-co-caprolactone), poly(D,L-Lactide-co-caprolactone-co-glycolide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes, polyalkylene oxides (PEO), polyalkylene terephthalates, polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), derivatized celluloses such as alkyl cellulose, hydroxy alkyl celluloses, cellulose ethers, cellulose esters, nitro celluloses, hydroxypropylcellulose, carboxymethylcellulose, polymers of acrylic acids, such as polymethylmethacrylate) (PMA), poly(ethyl(meth)acrylate), poly(butyl(meth)acrylate), poly (isobutyl (meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), polyiisobutyl acrylate), poly(octadecyl acrylate) (poly acrylic acids), polydioxanone, polyhydroxyalkanoates, polypropylene fumarate, polyoxymethylene, poloxamers, poly(ortho)esters, poly(butyric acid), poly(valeric acid), poly(lactide-co-caprolactone), trimethylene carbonate, polyvinylpyrrolidone, polyorthoesters, polyphosphazenes, and polyphosphoesters. In one embodiment, the nanoparticle or microparticle may comprise PLGA and/or PLGA-PEG polymers. In alternative embodiments, the particle carrier may be a micellar assembly comprising surfactants, such as a liposome. Various nanoparticle or microparticle carrier systems have been described, and find use with the invention, including those described in U.S. Pat. No. 8,206,747 B2, US 2014/0112881 A1, US 2015/0202163 A1, US 2015/0209447 A1, and WO 2015/105549 A2, all of which are hereby incorporated by reference in their entireties.

The nanoparticle or microparticle may be designed to provide desired pharmacodynamic advantages, including circulating properties, biodistribution, and degradation kinetics. Such parameters include size, surface charge, polymer composition, targeting ligand conjugation chemistry, among others. For example, in some embodiments, the particles have a PLGA polymer core, and a hydrophilic shell formed by the PEG portion of PLGA-PEG co-polymers. The hydrophilic shell may further comprise ester-end capped PLGA-PEG polymers that are inert with respect to functional groups.

The nanoparticles can be tuned for a specific biodegradation rate in vivo by adjusting the LA:GA ratio and/or molecular weight of the PLGA polymer. In some embodiments, the PLGA is based on a LA:GA ratio of from 20:1 to 1:20, including compositions of L/G of: 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. PLGA degrades by hydrolysis of its ester linkages. The time required for degradation of PLGA is related to the ratio of monomers: the higher the content of glycolide units, the lower the time required for degradation as compared to predominantly lactide units. In addition, polymers that are end-capped with esters (as opposed to the free carboxylic acid) have longer degradation half-lives. The molecular weights of the PLGA and PEG co-polymers allow for tunable particle size. For example, PLGA co-polymers may have a molecular weight within about 10 kDa to about 100 kDa, and PEG co-polymers may have a molecular weight within about 2 kDa to about 20 kDa.

The pharmaceutical composition can be targeted to the liver selectively over other tissues by incorporating an anionic functionality into the particle carrier, which targets OATP group of receptors. An exemplary anionic functionality is mercaptopropionic acid, and other carboxylates. Alternatively, or in addition, the pharmaceutical composition may comprise other targeting schemes to direct the particle carrier to target tissues or cells. Such targeting may improve the efficiency and effectiveness of the guest molecule, such as a TRβ agonist, as the local concentration of the guest molecule is elevated. In some embodiments, the targeting agent may be a tissue selective targeting agent, or may be selective for certain cells, such as but not limited to hepatocytes. Nanoparticle or microparticle carriers in these embodiments, which comprise a TRβ agonist may be used in a treatment of diseases and conditions related to TRβ function. Exemplary strategies for targeted drug delivery are described in Muro S., “Challenges in design and characterization of ligand-targeted drug delivery systems,” J. Control Release, 164(2): 125-37 (2012), which is incorporated by reference in its entirety.

In some embodiments, the targeting agent may be an antibody or antigen-binding fragment thereof. In other embodiments, the targeting agent may be a peptide, aptamer, adnectin, polysaccharide, or biological ligand. The various formats for target binding include a single-domain antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody; a Transbody, an Anticalin, an AdNectin, an Affilin, a Microbody, a peptide aptamer, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, a peptide mimetic molecule, or a synthetic molecule, or as described in US Patent Nos. or Patent Publication Nos. U.S. Pat. No. 7,417,130 B2, US 2004/132094 A1, U.S. Pat. No. 5,831,012 B2, US 2004/023334 A1, U.S. Pat. No. 7,250,297 B2, U.S. Pat. No. 6,818,418 A1, US 2004/209243 A1, U.S. Pat. No. 7,838,629 B2, U.S. Pat. No. 7,186,524 B2, U.S. Pat. No. 6,004,746 B2, U.S. Pat. No. 5,475,096 B2, US 2004/146938 A1, US 2004/157209 A1, U.S. Pat. No. 6,994,982 B2, U.S. Pat. No. 6,794,144 B2, US 2010/239633 A1, U.S. Pat. No. 7,803,907 B2, US 2010/119446 A1, and/or U.S. Pat. No. 7,166,697 B2, all of which are hereby incorporated by reference in their entireties. See also, Storz MAbs. 2011 May-June; 3(3): 310-317, which is incorporated by reference in its entirety. Exemplary targeting agents include antigen-binding antibody fragments, such as but not limited to F(ab′)2 or Fab, a single chain antibody, a bi-specific antibody, or a single domain antibody.

In certain embodiments, the targeting agent is triantennary N-Acetylgalactosamine (GalNAc), dimeric GaINAc or monomeric GalNAc, which targets the particle carriers to hepatocytes. Alternative targeting agents may bind integrins (e.g., RGD peptide), and in some embodiments may be a cell-penetrating peptide (CPP) or an anionic functionality (carboxylate such as mercaptopropionic acid) that targets the OATP group of receptors.

The targeting agent can be chemically conjugated to the particles using any available process. Functional groups for conjugation include COOH, NH₂, and SH. See, for example, Hermanson, BIOCONJUGATE TECHNIQUES, Academic Press, New York, 1996, which is incorporated by reference in its entirety. Activating functional groups include alkyl and acyl halides, amines, sulfhydryls, aldehydes, unsaturated bonds, hydrazides, isocyanates, isothiocyanates, ketones, and other groups known to activate for chemical bonding. Alternatively, the targeting agent can be conjugated through the use of a small molecule-coupling reagent. Non-limiting examples of coupling reagents include carbodiimides, maleimides, N-hydroxysuccinimide esters, bischloroethylamines, bifunctional aldehydes such as glutaraldehyde, anhydrides, and the like.

Additional cellular targets and potential target tissues and cells are summarized in Table 1.

TABLE 1
Targeting Schemes

| Targeted Molecule | Exemplary Target Tissue/Cells |
|---|---|
| ACE | Endothelial cells (e.g., lung endothelial |
| | cells) |
| Aminopeptidase N | Vascular cells |
| Aminopeptidase P | Endothelial cells (e.g., lung endothelial |
| | cells) |
| αvβ 3 & αvβ 5 integrins | Vascular cells |
| B-lymphocyte antigen CD20 | B cells |
| Endoglin | Endothelial cells (e.g., lung endothelial |
| | cells) |
| ErbB2 | Breast and ovary |
| Folate receptor | Cancer and immune cells |
| Glycosylated molecules | Various types |
| gp60 | Vascular cells |
| ICAM-1 | Immune cells |
| IL-2 receptor | Cancer and immune cells |
| IGF-1 receptor | Cancer cells |
| Insulin Receptor | Hepatocytes and adipocytes |
| LDL receptor family | Blood and immune cells |
| LHRH receptor | Prostate cells |
| LFA-1 | Lymphocytes |
| Mannose-6 phosphate receptor | Immune cells |
| MMPs | Cancer and immune cells |
| MUC1 | Breast and bladder cells |
| PECAM-1 | Immune cells |
| Selectins | Vascular cells (e.g., in solid tumors) and |
| | immune cells |
| Transferrin receptor | Cancer cells and blood-brain-barrier |
| VCAM-1 | Vascular cells (e.g., in solid tumors) and |
| | immune cells |
| VEGF receptor | Vascular cells (e.g., in solid tumors). |

In some embodiments, the targeting agent may be conjugated or attached to the surface of the particle carrier. In some embodiments, the targeting agent is an antibody or antibody fragment linked to the polymeric units on the surface of the nanoparticle or microparticle, either non-covalently or covalently. In some embodiments, the antibody or other targeting ligand is covalently conjugated to the terminus of PEG or OEG chains using known processes.

In some embodiments, the particle carrier is targeted to the liver, kidney, lung, heart, nerves, macrophages, hematopoietic stem cells, hepatic stellate cells, vasculature, brain, vagina, uterus, stomach, intestine (small and large intestine), or muscles of specific organs. In certain embodiments, the guest molecule is a TRβ agonist or antagonist, and is targeted to a cell or tissue selected from hepatocytes, vasculature, smooth muscles (e.g., smooth muscles associated with bronchoconstriction or smooth muscles associated with gastrointestinal tract), kidney, immune cells, stomach, uterus (or smooth muscle of the uterus), or neuronal cells such as but not limited to peripheral nerves.

In still other embodiments, the particle carrier may be directed by passive targeting, referring to the accumulation of the particle into particular regions of the body due to the natural features and physiological role of the tissues and cells. Thus, in some embodiments, the particle carrier may accumulate in the desired tissues or cells in the absence of a targeting agent. For example, the particle carrier may accumulate in organs of the reticulo-endothelial system (RES), such as but not limited to the liver and/or the spleen, which may capture foreign substances and objects that reach the systemic circulation. In some embodiments, the particle carrier may accumulate in the monocyte/macrophage system. In another embodiment, the particle carrier may accumulate in the vasculature of tumors, which show an enhanced permeability and retention effect. In some embodiments, the particle carrier is accumulated in liver, kidney, and/or lung.

The pharmaceutical composition may be formulated into liquid or solid dosage forms and administered systemically or locally. Techniques for formulation and administration may be found in Remington: The Science and Practice of Pharmacy (20th ed.) Lippincott, Williams & Wilkins (2000), which is incorporated by reference in its entirety. Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery. In some embodiments, the pharmaceutical composition is formulated for parenteral or enteral administration.

While the form and/or route of administration can vary, in some embodiments the pharmaceutical composition is administered parenterally (e.g., by subcutaneous, intravenous, or intramuscular administration). For injection, the agents of the disclosure may be formulated and diluted in aqueous solutions, such as in physiologically compatible buffers such as Hank's solution, Ringer's solution, or physiological saline buffer.

In some embodiments employing oral administration or administration to the GI, the pharmaceutical composition may be formulated to comprise an enteric coating. The enteric coating controls the release of the nanoparticles to avoid harsh environments of the stomach for example, by employing a coating that is insoluble at low pH, but soluble at higher pH so as to release particle carriers in the small or large intestine.

In one aspect, the invention relates to using the pharmaceutical composition described herein to treat diseases and conditions associated with Thyroid hormone receptor functions (e.g., TRβ) functions, such as but not limited to liver disease.

In exemplary embodiments, the invention provides a pharmaceutical composition comprising a particle carrier non-covalently encapsulating an effective amount of a TRβ agonist. The particle carrier comprises an anionic functionality that targets OATP group of receptors (which in some embodiments is a carboxylate functionality), and the particle carrier comprises a disulfide-crosslinked polymeric interior that is not substantially degraded in normal blood plasma and is substantially degraded in the presence of intracellular concentrations of GSH. In exemplary embodiments, the anionic functionality is incorporated in the form of mercaptopropionic acid. In some embodiments, the TRβ agonist is axitirome. In such embodiments, the particle carrier has an average diameter in the range of about 10 nm to about 200 nm, or in the range of about 20 nm to about 100 nm, or in the range of about 25 nm to about 75 nm, and has a zeta potential in the range of about −5 mV to about −40 mV or in the range of about −10 mV to about −30 mV, or about −15 mV to about −25 mV. The particle carrier is formed by self-assembly in an aqueous environment. For example, the particle carrier is formed in the presence of the TRβ agonist axitirome and an amphiphilic copolymer. The amphiphilic copolymer comprises hydrophilic OEG branch groups (as described) and disulfide-linked hydrophobic branch groups (e.g., PDS moieties) to drive micellar assembly and agonist encapsulation, followed by cross-linking of hydrophobic branch groups through disulfide exchange reactions. The disulfide exchange reaction shuffles sulfhydryl groups of dithiothreitol, for example, into the disulfides of disulfide-linked hydrophobic branch groups. In various embodiments, the OEG branch groups and the hydrophobic branch groups are present at a ratio of from about 1:4 to about 4:1. The amphiphilic co-polymer can be prepared by RAFT polymerization of PDSEMA and oligoethylene glycol monomethyl ether methacrylate. In various embodiments, the particle carrier has a crosslinking density from about 10% to 70%, or from about 20% to about 60%, or from about 30% to about 50% with respect to total number of disulfide-containing structural units in the polymer, and a density of anionic ligand of about 20% to about 60%, or about 30% to about 50% with respect to total number of structural units in the polymer. In some embodiments, the density of the anionic ligand is about 35% to about 45% (e.g., about 40%) with respect to total number of structural units in the polymer.

In another aspect, the present invention relates to a method for making the pharmaceutical composition described herein. The method comprises incorporating the TRβ agonist or antagonist into a particle carrier, including by cross-linking of hydrophobic branch groups as described above, or by nanoprecipitation using PLGA-PEG polymers or similar polymer constructs.

The TRβ agonist or antagonist, is released upon partial or complete degradation or de-crosslinking of polymer molecules at or near the biological site. For example, after transport of the particle carrier to the target tissue or cells, the carrier may be degraded or de-crosslinked, thereby releasing the active agent. In one embodiment, the degradation is triggered by an endosomal or intracellular environment upon cell internalization. For example, the degradation may be caused by breaking the disulfide bonds in the particle carrier in a reducing environment. Alternatively, degradation of the particle carrier may be triggered by low pH. In some embodiments, the active agent is not substantially released at concentrations of reducing agent characteristic of blood plasma, so that active agent is only released after cell internalization.

In other aspects, the invention provides a method for treating a disease or condition, comprising administering an effective amount of the pharmaceutical composition described herein to a patient in need of treatment. In various embodiments, the pharmaceutical composition is administered by intravenous or intraarterial administration, oral administration, intramuscular administration, or subcutaneous administration. In some embodiments, the composition is administered parenterally, such as by intravenous infusion or subcutaneous administration.

In yet another aspect, the present invention relates to a nanogel comprising a crosslinked copolymer and a thyroid hormone receptor-β (TRβ) agonist or a TRβ antagonist encapsulated in the crosslinked polymer.

In certain embodiments of the nanogel, the crosslinked copolymer comprises structural units of:

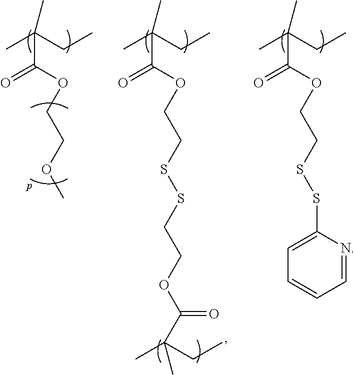

and

In certain embodiments, the crosslinked copolymer comprises the structural formula:

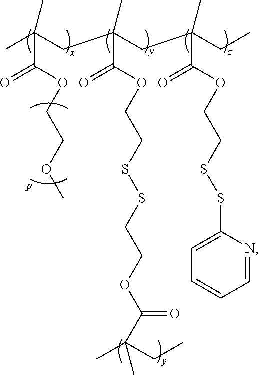

wherein each of x, y and z is independently a positive integer in the range from 1 to about 100 (e.g., about 10 to about 100, or about 20 to about 80, or about 40 to about 80). It is understood that the order of monomers (e.g., structural units) in the polymer is essentially random.

In certain embodiments of the nanogel, the crosslinked copolymer further comprises a targeting moiety adapted to accumulate in a target tissue or organ.

In certain embodiments, the target tissue or organ is liver.

In certain embodiments, the targeting moiety comprises a carboxylate.

In certain embodiments, the targeting moiety comprises the structural unit of:

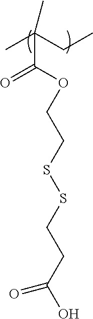

In certain embodiments, the crosslinked copolymer comprises the structural units of the following structural formula:

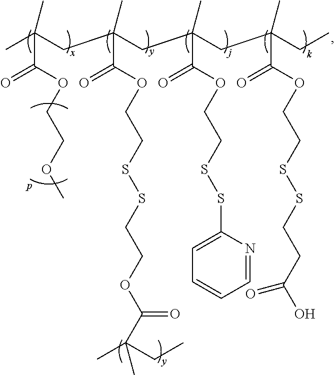

wherein j is a percentage of (x+y+j+k) in the range from 0% to about 70% (e.g., 10% to 50%, or 10% to 40%, or 10% to 30%, or 10% to 20%, or 20 to 40%), x and k are independently in the range from 1% to about 50% (e.g., independently 10% to 50%, or 20% to 50%, or 30% to 50%).

In certain embodiments of the nanogel, the TRβ agonist or TRβ antagonist is axitirome (CGS26214), Triiodothyronine (T3), Thyroxine (T4), Sobetirome (GC-1), Eprotirome (KB2115), Resmetirom (MGL-3196), VK2809, 1S25, TG68, or CGS23425.

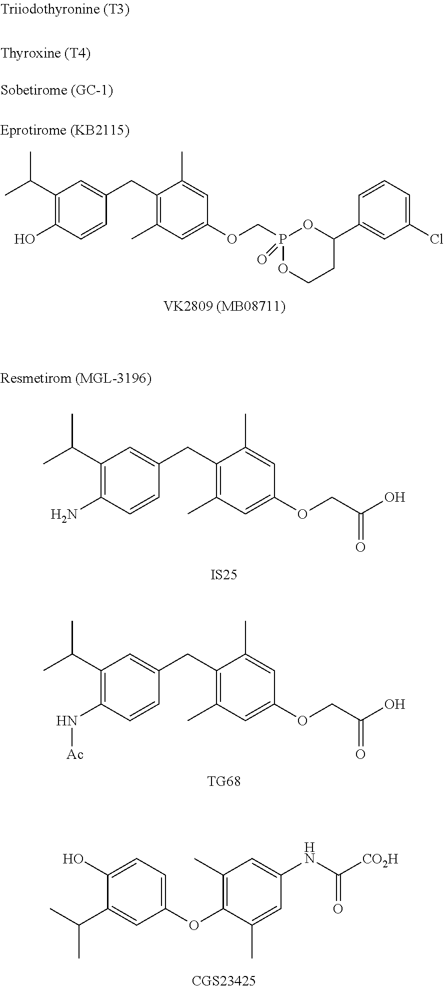

In certain embodiments, the crosslinked copolymer is characterized by a crosslinking density in the range from about 10% to about 70% (e.g., from about 20% to about 60%, about 30% to about 50%).

In certain embodiments, the nanogel is in the form of nanoparticles having an average diameter in the range from about 10 nm to about 200 nm (e.g., about 20 nm to about 100 nm).

In yet another aspect, the present invention relates to a pharmaceutical composition comprising the nanogel disclosed herein.

In certain embodiments, the pharmaceutical composition further comprises a pharmaceutically acceptable excipient, carrier, or diluent.

In yet another aspect, the present invention relates to a method for treating a disease or condition, comprising administering to a patient in need thereof an effective amount of a pharmaceutical composition disclosed herein.

In certain embodiments of the method, the disease or condition is selected from the group consisting of NAFLD, NASH, hypercholesterolemia, hyperlipidemia, metabolic syndrome, and obesity, or a related disease or condition.

In some embodiments, the patient has NAFLD. In some embodiments, the patient has NASH or alcoholic steatohepatitis (ASH). In some embodiments, the patient has liver fibrosis. In other embodiments, the patient to be treated is a liver transplant recipient or liver transplant donor. In still other embodiments, the patient to be treated may have hepatocellular carcinoma (HCC). In some embodiments, the patient to be treated may have type 1 or type 2 diabetes, or metabolic syndrome. For example, the patient to be treated may be obese or overweight. In some embodiments, the patient may be hypercholesterolemic or hyperlipidemic. In such embodiments, the TRβ agonist stimulates liver metabolism, such as fatty acid β-oxidation and oxidative phosphorylation in hepatocytes. The method can result in one or more of: lowering of total serum cholesterol, lowering LDL cholesterol, lowering of serum triglycerides, lowering of serum lipoprotein A, decrease to in hepatic fat, increase in lipolysis, increase in hepatocyte proliferation, and weight loss.

In still other embodiments, the particle carrier is targeted to the central nervous system (e.g., for delivery to a patient having a demyelinating disorder such as multiple sclerosis), and encapsulates a TRβ agonist. In these embodiments, the method can result in increased myelin repair.

In still other embodiments, the carrier encapsulates a TRβ antagonist, for delivery to any desired cell or tissue (including but not limited to the liver, heart, or CNS), to reduce symptoms of thyrotoxicosis. In some embodiments, the patient has Grave's disease, toxic multinodular goiter, toxic adenoma, TSH-producing adenoma or pituitary adenoma, HCG-mediated hyperthyroidism, thyroiditis, drug-induced increased secretion of thyroid hormone (e.g., induced by amiodarone or iodinated contrast), factitious hyperthyroidism, or excessive replacement therapy.

As used herein, the term “patient” refers to any animal (e.g., a mammal), including, but not limited to, humans, non-human primates, rodents, and the like, which is to be the recipient of a particular treatment. In various embodiments, the patient is a human.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique. Those in need of treatment include those already with the disorder as well as those prone to have the disorder or those in whom the disorder is to be prevented.

As used herein, the term “effective amount” of an active agent or composition thereof refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient. For example, the pharmaceutical composition described herein may be administered in a dose of about 1 μg/kg to about 10 mg/kg, or from about 5 μg/kg to about 1 mg/kg, or from about 10 μg/kg to about 500 μg/kg, or from about 50 μg/kg to about 200 μg/kg, where kg is the body weight of the patient to be treated.

As used herein, the term “pharmaceutically acceptable excipient, carrier, or diluent” refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

In various embodiments, the pharmaceutical composition is administered from once daily to about once monthly. In some embodiments, the composition is administered about weekly or about every other week. In still other embodiments, the composition is administered about every other month (e.g., about 6 times per year) or about quarterly (e.g., about 4 times per year).

In some embodiments, the patient is a liver transplant recipient, a liver transplant donor, or the patient has cirrhotic liver disease, alcoholic liver disease, liver fibrosis, or acute liver failure.

In some embodiments, the patient has an acute liver failure due to a chemical toxicity. For example, the chemical toxicity may be due to acetaminophen administration or overdose.

In some embodiments, the patient has type 1 or type 2 diabetes or metabolic syndrome. In some embodiments, the patient has elevated cholesterol, elevated triglycerides, or hyperlipidemia. For example, the patient is obese or overweight. In some embodiments, the patient has insulin resistance.

As used herein, the term “about” means±10% of an associated numerical value, unless the context requires otherwise.

### EXAMPLES

Various embodiments of the present invention can be better understood by reference to the following Examples that are offered by way of illustration. The present invention is not limited to the Examples given herein.

### Example 1. Synthesis of PEG:PDS Random Copolymers

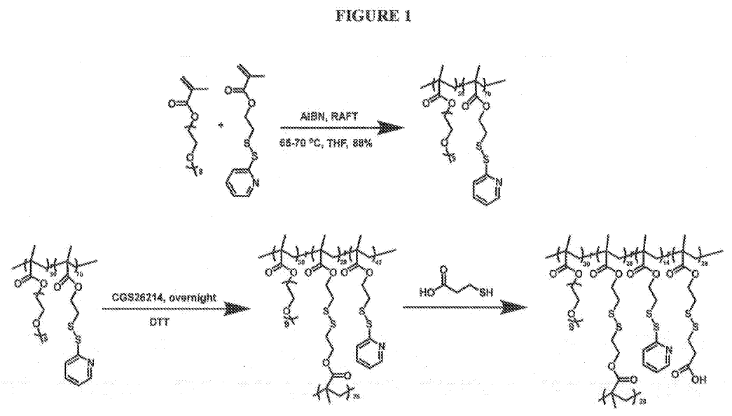

Random copolymers containing polyethylene glycol monomethyl ether methacrylate and pyridinyldisulfide ethyl methacrylate (PDSEMA) as side chain functionalities were synthesized using RAFT polymerization as previously reported (Ryu, J. H.; Chacko, R. T.; Jiwpanich, S.; Bickerton, S.; Babu, R. P.; Thayumanavan, S. Self-Cross-Linked Polymer Nanogels: A Versatile Nanoscopic Drug Delivery Platform. Journal of the American Chemical Society 2010). Briefly, a mixture of PDSEMA (595.84 mg, 2.27 mmol), polyethylene glycol monomethyl ether methacrylate (average MW: 500, 500 mg, 1 mmol), 4-cyano-4-[(dodecyl-sulfanylthiocarbonyl)sulfanyl] pentanoic acid (27.05 mg, 0.067 mmol) and AIBN (2.2 mg, 0.0134 μmol) was dissolved in anhydrous THF (2.2 mL) and degassed by performing three freeze-pump-thaw cycles. The reaction mixture was sealed and then put into a pre-heated oil bath at 70° C. for 10 h. To remove unreactive monomers, the resultant mixture was precipitated and washed in cold diethyl ether for several times to yield the random copolymer as a yellow gel liquid. See FIG. 1.

¹H-NMR spectra were recorded on a 400 MHz Bruker NMR spectrometer using the residual proton resonance of the solvent as the internal standard. Molecular weights of the polymers were estimated by gel permeation chromatography (GPC, waters) using PMMA standard with a refractive index detector. The size of polymers was detected by Dynamic light scattering (DLS) measurements using a Malvern Nanozetasizer.

### Example 2. Preparation of CGS26214-Encapsulated Nanogels with Anionic Group-Modified Backbone (CGS-ANG) and Empty Nanogels (BNG)

Nanogels were prepared by chemically cross-linking the equilibrium assembly of the polymers or drug-encapsulated nanoassemblies at 25° C. using DTT as a reducing agent as previously reported (Ryu, J. H.; Chacko, R. T.; Jiwpanich, S.; Bickerton, S.; Babu, R. P.; Thayumanavan, S. Self-Cross-Linked Polymer Nanogels: A Versatile Nanoscopic Drug Delivery Platform. Journal of the American Chemical Society 2010). To prepare CGS-ANG, PEG:PDS random copolymers (50 mg) were dissolved in PBS buffer (5 mL) upon which CGS26214 (0.25 mg dissolved in 150 μL THF) was added into the polymer solution to form drug-encapsulated nanoassemblies. The mixture was stirred overnight at room temperature, open to the atmosphere and thereby allowing the organic solvent to evaporate. Calculated amount of DTT (3.11 mg) was then added to the mixture and stirred for another 24 hours to generate cross-linked drug-encapsulated nanogels. To prepare empty nanogel (BNG), PEG:PDS random copolymers (50 mg) were dissolved in PBS buffer (5 mL) and calculated amount of DTT (3.11 mg) was then added to the mixture and stirred for 24 hours to generate cross-linked empty nanogels. Cross-linking degree was determined by calculating the amount of byproduct 2-pyridinethione that was produced using vis-UV spectroscopy (molar extinction coefficient=8.08×10³ M⁻¹ cm⁻¹ at 343 nm). Crosslinking percentage was calculated by assuming that formation of a single, crosslinked disulfide bond would require cleavage of two PDS units and produce two pyridothione molecules. The size and zeta potential were then measured by dynamic light scattering at 0.2 mg/mL nanogel concentration.

To modify CGS26214-encapsulated nanogels and empty nanogels with anionic ligands, 3-mercaptopropionic acid (4.29 mg) was added to the nanogel solution and stirred overnight. Anionic ligand percentage was determined by calculating the amount of 2-pyridinethione released, since formation of a single anionic ligand will require cleavage of one PDS unit and produce one pyridothione molecule. FIG. 1 shows the synthetic scheme for production of liver targeted, CGS26214-encapsulated nanogel.

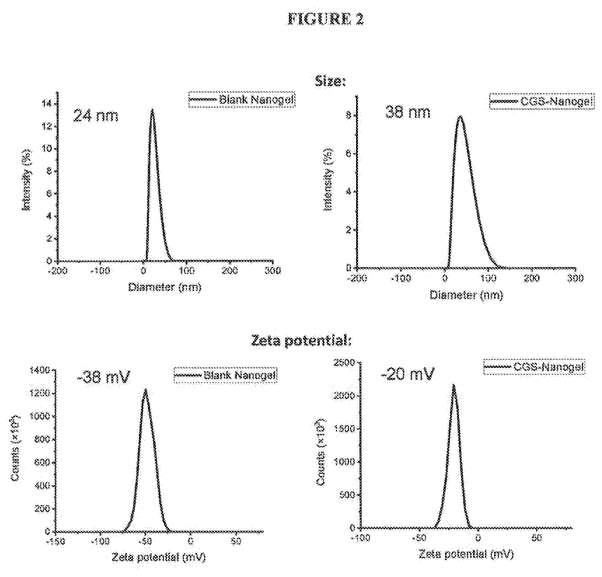

The resulting nanogels were purified by dialysis against PBS buffer (cutoff MW=3500 Da) for 3 days and sterile filtered through 0.22 μm Millipore PVDF filters to remove free drug and other small molecule reactants. The size and zeta potential were then measured by DLS at 0.2 mg/mL nanogel concentration. FIG. 2. For Transmission Electron Microscope (TEM) study, 20 mg/mL sample was dropped onto carbon-coated copper grid and was dried by slow evaporation in air overnight. Images were recorded on a JEOL-2000FX electron microscopy operated at 200 kV and at a nominal magnification of 5000×. At least 10 locations on the TEM grid were examined.

### Example 3. Analysis of CGS26214 Loading Efficiency

CGS-ANG solution (0.5 mL, nanogel concentration of 10 mg/mL) was degraded by adding high concentration of DTT (155 mg) and stirred for 8 h. The solution was lyophilized for 8 h and the product was reconstituted in methanol for further analysis.

Quantitative determination of CGS26214 released from the nanogel polymer was conducted by a sensitive liquid chromatography-tandem mass spectrometry (LC-MS/MS) method (Majumdar, T. K.; Wu, S.; Tse, F. L. S. Quantitative Determination of CGS 26214, a Cholesterol Lowering Agent, in Human Plasma Using Negative Electrospray Ionization Liquid Chromatography-Tandem Mass Spectrometry. Journal of Chromatography B: Biomedical Sciences and Applications 2001). Briefly, sample was hydrolyzed by mixing with 2 mL of a freshly prepared aqueous solution of 0.5 M ammonium hydroxide and kept on the bench for 60 min at room temperature. The hydrolysate was treated with 0.4 mL of glacial acetic acid to make the content slightly acidic (pH 4-5). The sample was filtered through 0.22 μm filters and dried in a Savant evaporator at room temperature. The residue was reconstituted in methanol and diluted for 20 times for LC-MS/MS quantification.

Sample chromatography was performed on an Acquity UPLC system (Water Corp., Milford, MA, USA) with a temperature controlled autosampler set to 4° C. Separation was performed at room temperature on ZORBAX Stable Bond Aq reversed phase analytical column (4.6 mm×250 mm, 5 μm particle size, 80 Å; Agilent Technologies, Inc., Santa Clara, CA). A C18 guard cartridge (4.6 mm/12.5 mm, 5-Micron, Agilent) was used to protect the main column. An isocratic flow was used to elute the analytes from the column. The mobile phases consisted of methanol-water-5 M ammonium hydroxide methanol-water (60:55:5, v/v) at a rate of 0.2 mL/min. The column temperature was maintained at 45° C. and the injection volume was 5 μL with a total run time of 20 min. On-line Mass Spectrometry (MS) detection was performed on a Xevo TQ-S tandem quadrupole mass spectrometers (Water Corp., Milford, MA, USA) equipped with an electrospray ionization (ESI) source coupled to the UPLC system. Experiments were performed in the negative ionization mode of detection. Nitrogen was used as the de-solvation gas and argon gas used for collision-induced dissociation (CID). Hydrolyzed CGS26214 was fragmented with cone voltage and collision energy set at 35V and 22V. Experiments were performed in the selected reaction monitoring (SRM) mode to detect precursor to product ion transitions of m/z 424 [M2H]→352. SRM chromatograms were quantified using MassLynx v 4.1 software (Waters Inc) by integration of peaks.

Standard solution of hydrolyzed product from CGS26214 was prepared prior to experiments by diluting with methanol. Final concentrations of standards were in the range between 5 and 160 ng/mL. Calibration curve was constructed by plotting the ratio of the peak area (from LC-MS/MS) of the spike analyte at each concentration. The concentration of CGS26214 sample was determined from these standards.

An exemplary batch of drug loaded nanogel had the following properties: Drug loading percentage of 0.12%; drug encapsulation efficiency of 24%; percent crosslinking of 40%; and anionic ligand density (for liver targeting) of 40%.

### Example 4. Animals and Experimental Design

All animal care and experimental procedures were approved by the Ethical Committee on Animal Care and Experimentation. Male C57BL/6J mice were purchased at 6 weeks of age from the Jackson Laboratory and housed in a controlled environment (12 h light/dark cycle, 21±2° C., humidity 50±10%). Mice were permitted ad libitum access to water and either 10 kcal % fat control diet (CD, Cat #D09100304, Research Diets) or 40 kcal % fat, 20 kcal % fructose and 2% cholesterol high fat diet (HFD, Cat #D09100310, Research Diets).

To test the preventative effect of CGS-ANG in treating HFD-induced NASH, mice were fed high fat diet (HFD) or low fat diet (CD) for 12 weeks (mild NASH model) and then randomly assigned to 9 groups with 8-10 mice per group before treatment: (1), CD: mice fed the CD diet and treated with vehicle; (2), HFD: mice fed the HFD diet and treated with vehicle; (3), CGS-D1: mice fed the HFD diet and treated with 10 μg/kg of CGS26214 (suspended in saline with 1% DMSO); (4), CGS-D2: mice fed the HFD diet and treated with 20 μg/kg of CGS26214; (5) CGS-D3: mice fed the HFD diet and treated with 60 μg/kg of CGS26214; (6) CNG-D1: mice fed the HFD diet and treated with CGS-ANG loaded with CGS26214 at a dose of 10 μg/kg; (7) CNG-D2: mice fed the HFD diet and treated with CGS-ANG loaded with CGS26214 at a dose of 20 μg/kg; (8) CNG-D3: mice fed the HFD diet and treated with CGS-ANG loaded with CGS 26214 at a dose of 60 μg/kg; (9) BNG: mice fed the HFD diet and treated with BNG with the same dose of nanogels with group (8). All the treatments were injected intraperitoneally at the doses indicated once per day before the dark cycle of the day for 5 weeks.

To test the therapeutic effect of CGS-ANG in treating HFD-induced NASH, mice were fed HFD or LFD for 24 weeks (fully developed NASH model) and then randomly assigned to 9 groups with 8-10 mice per group before the medical treatments: (1), CD: mice fed the CD diet and treated with vehicle; (2), HFD: mice fed the HFD diet and treated with vehicle; (3), CGS-D1: mice fed the HFD diet and treated with 5 μg/kg of CGS26214; (4), CGS-D2: mice fed the HFD diet and treated with 10 μg/kg of CGS26214; (5) CGS-D3: mice fed the HFD diet and treated with 20 μg/kg of CGS 26214; (6) CNG-D1: mice fed the HFD diet and treated with CGS-ANG loaded with CGS26214 at a dose of 5 μg/kg; (7) CNG-D2: mice fed the HFD diet and treated with CGS-ANG loaded with CGS 26214 at a dose of 10 μg/kg; (8) CNG-D3: mice fed the HFD diet and treated with CGS-ANG loaded with CGS 26214 at a dose of 20 μg/kg; (9) BNG: mice fed the HFD diet and treated with BNG with the same dose of nanogels as group (8). All the treatments were injected intraperitoneally at the doses indicated once per day before the dark cycle of the day for 5 weeks.

Body weight and food intake per cage were measured regularly during the study. Feces collections were done before, in the middle of and at the end of treatments. Feces from individually housed mice were collected on each day of the 3-day feeding period.

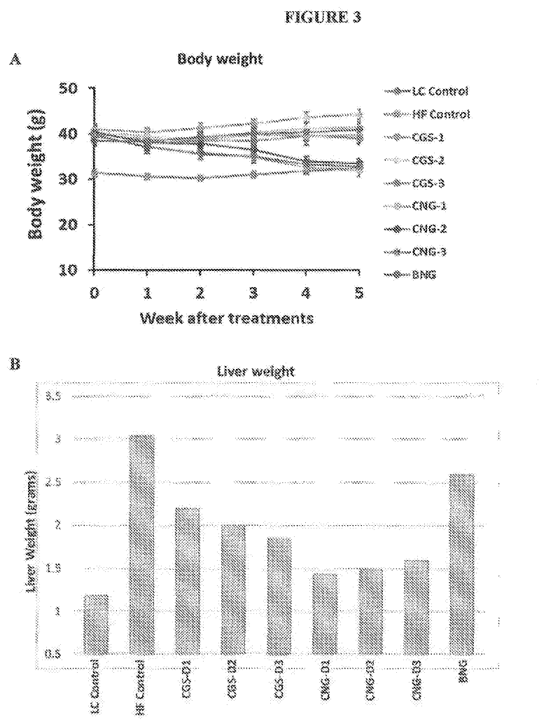

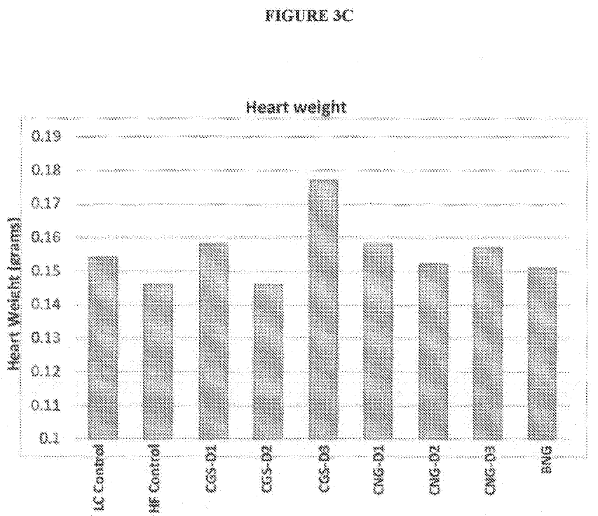

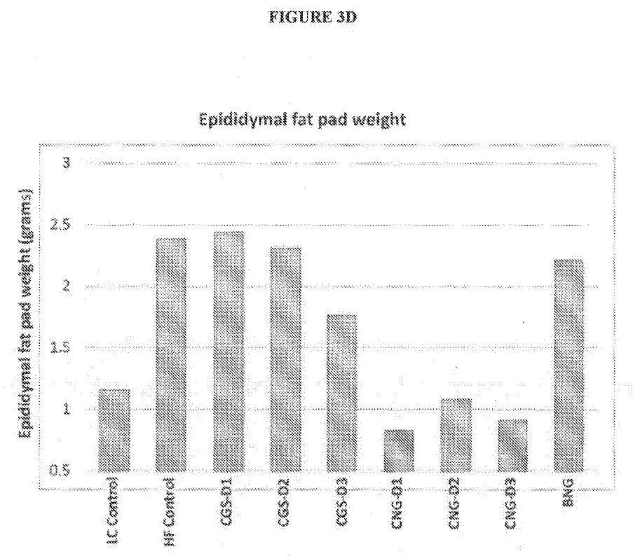

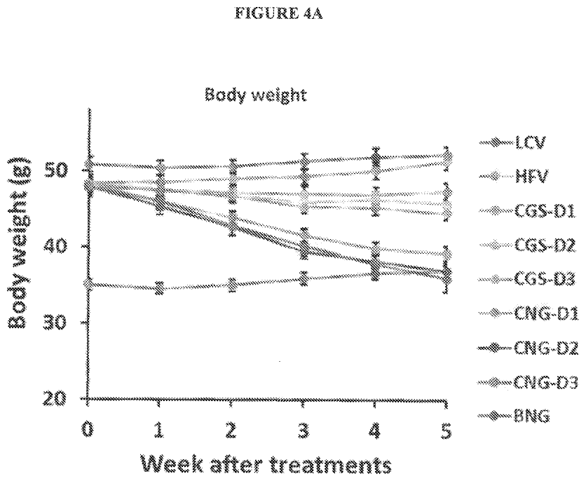

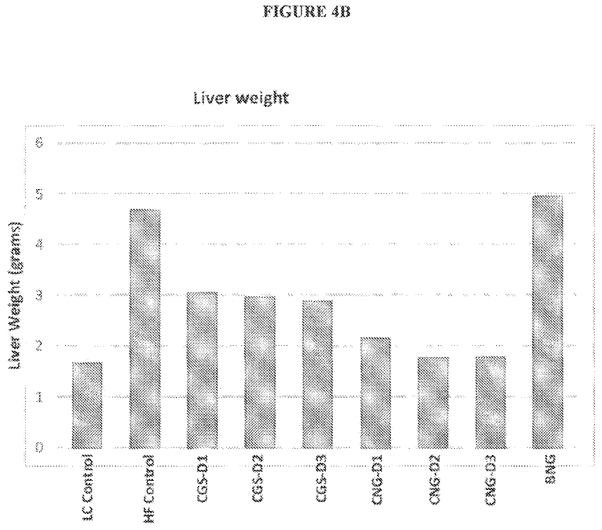

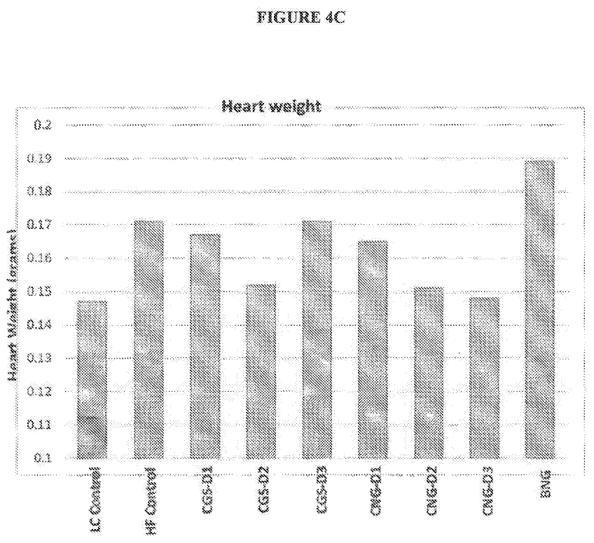

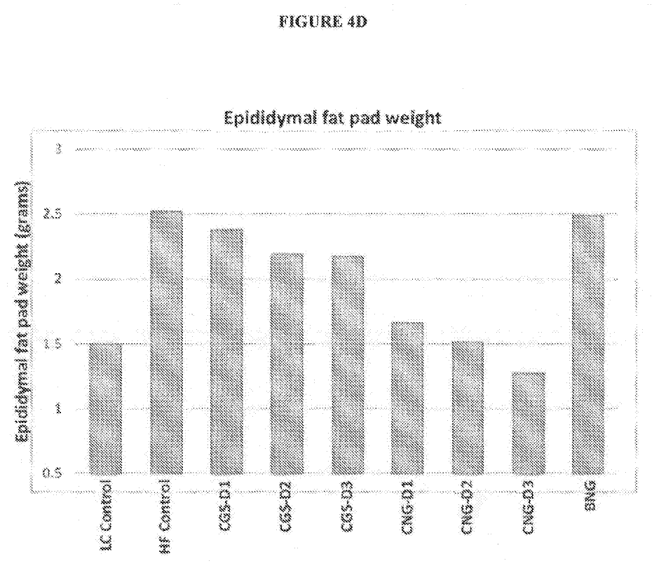

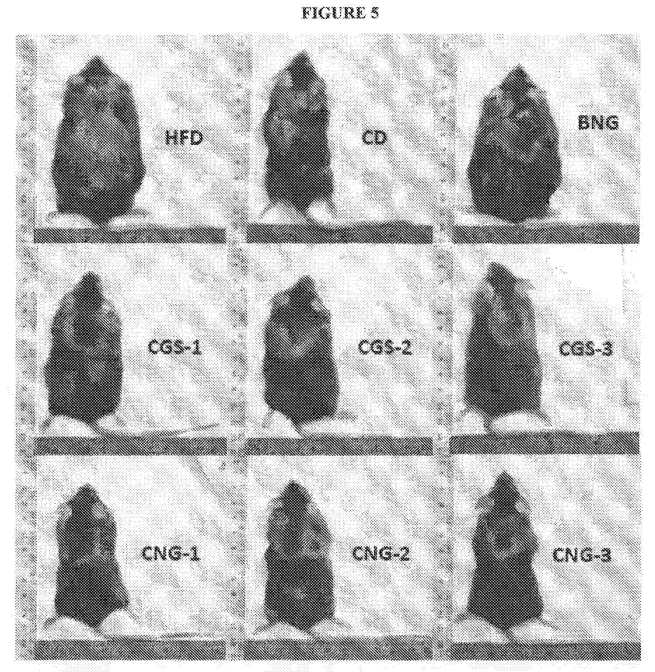

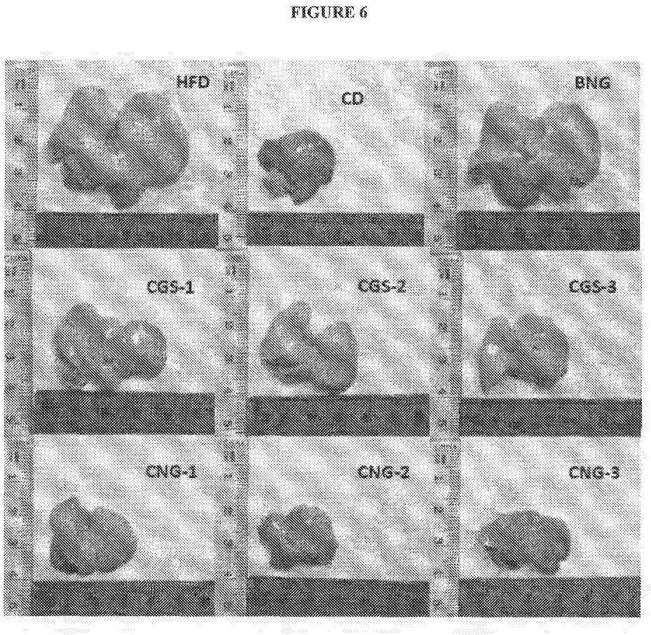

After each designated treatment period, all the mice were sacrificed unfasted by cardiac puncture after gradual-fill CO₂ asphyxiation. Terminal blood samples were collected in SST-SERUM separator tubes for serum collection. Epididymal fat pads (EFP), liver and hearts were removed and weighed. Data (weight gain, liver weight, heart weight, epididymal fat pad weight) from the 12 week and 24 week study are shown in FIGS. 3A-3D and 4A-4D, respectively. Portions of livers and hearts were collected and stored at −80° C. or fixed in 4% paraformaldehyde solution (Sigma-Aldrich, St. Louis, MO) for further analysis. Visual images of mice from each treatment groups at 24 weeks is shown in FIG. 5 while images of mice liver from each treatment group at 24 weeks is shown in FIG. 6.

As illustrated in FIGS. 5 and 6, CGS26214 (unencapsulated) showed only moderate effects on body weight and liver weight in the two models, while gel-encapsulated CGS26214 showed dramatic improvements. In fact, body weight for gel-encapsulated drug treatment group was not statistically different from the low-fat diet control.

### Example 5. Serum and Liver Biochemical Analysis

Analysis of serum cholesterol (TC), high-density lipoprotein cholesterol (HDL-C), low-density lipoprotein cholesterol (LDL-C), triglycerides (TG), serum alanine aminotransferase (ALT) and aspartate aminotransferase (AST) was performed at Indexx BioAnalytics (North Grafton, MA) Plasma glucose, insulin, amyloid A (SAA), E-selectin and monocyte chemoattractant protein-1 (MCP-1) were quantified using commercial kits. Data from the 12 week and 24 week study are shown in Table 2 and Table 3, respectively.

TABLE 2
Effect of treatment of CGS26214 and CGS26214-encapsulated nanogel in mild NASH model (12 week of HFD/CD prior to dosing)*

| | LC | HF | | | | | | | |
|---|---|---|---|---|---|---|---|---|---|
| | Control | Control | CGS-1 | CGS-2 | CGS-3 | CNG-1 | CNG-2 | CNG-3 | BNG |
| ALT | 33 ± 8 | 352 ± 54 | 196 ± 26 | 217 ± 28 | 279 ± 68 | 82 ± 16 | 95 ± 19 | 170 ± 20 | 233 ± 33 |
| AST | 76 ± 10 | 284 ± 42 | 205 ± 27 | 252 ± 47 | 303 ± 49 | 139 ± 7 | 128 ± 15 | 220 ± 25 | 228 ± 33 |
| Cholesterol | 161.8 ± 7.2 | 376.7 ± 19 | 158.2 ± 19.2 | 155.8 ± 11.8 | 105.5 ± 23.7 | 65 ± 10.4 | 90.5 ± 9.2 | 90.5 ± 11.6 | 265 ± 18.3 |
| (mg/dL) | | | | | | | | | |
| HDL | 84.2 ± 2.2 | 125.8 ± 1.0 | 87.2 ± 9.6 | 76.8 ± 5.8 | 53.1 ± 10.8 | 37.2 ± 2.5 | 45.0 ± 2.8 | 42.8 ± 5.6 | 117.8 ± 2.1 |
| Cholesterol | | | | | | | | | |
| (mg/dL) | | | | | | | | | |
| LDL | <7 | 37.8 ± 3.9 | <7 | <7 | <7 | <7 | <7 | <7 | 20.3 ± 2.4 |
| Cholesterol | | | | | | | | | |
| (mg/dL) | | | | | | | | | |
| Triglycerides | 84 ± 4.7 | 122 ± 9.6 | 87.6 ± 3.5 | 83.5 ± 10 | 78.7 ± 7.2 | 79 ± 4.3 | 105.5 ± 14 | 91.5 ± 7.9 | 93 ± 2.4 |
| (mg/dL) | | | | | | | | | |

*Data are shown as means ± SE of 8-10 animals/group.

TABLE 3
Effect of treatment of CGS26214 and CGS26214-encapsulated nanogel in fully developed NASH model (24 week of HFD/CD) prior to dosing)*

| | LC | HF | | | | | | | |
|---|---|---|---|---|---|---|---|---|---|
| | Control | Control | CGS-1 | CGS-2 | CGS-3 | CNG-1 | CNG-2 | CNG-3 | BNG |
| ALT | 70 ± 11^{a} | 482 ± 44^{b} | 276 ± 20^{d} | 257 ± 20^{cd} | 225 ± 10^{cd} | 172 ± 18^{acd} | 149 ± 14ª^{c} | 177 ± 36^{acd} | 479 ± 23^{b} |
| AST | 123 ± 8^{a} | 436 ± 34^{b} | 319 ± 15^{ceg} | 342 ± 316^{bceg} | 247 ± 17^{de} | 293 ± 30 [symbol] ^{fg} | 199 ± 12ª^{df} | 259 ± 55^{aeg} | 431 ± 32^{bc} |
| Choles- | 189.5 ± 8.1^{ad} | 445.2 ± 16.4^{b} | 278.9 ± 20.2^{cef} | 236.8 ± 16.6^{cd} | 196.9 ± 10.2^{ade} | 208 ± 9.1ª [symbol] | 130.9 ± 11.6^{e} | 145.1 ± 20.9ª | 374.8 ± 18.9^{b} |
| terol | | | | | | | | | |
| (mg/ | | | | | | | | | |
| dL) | | | | | | | | | |
| HDL | 86 ± 3.2ª | 135.3 ± 2.1^{b} | 116 ± 4.4 [symbol] | 113.4 ± 5.5^{ab} | 96.3 ± 3.9^{ac} | 101.5 ± 5.3 [symbol] | 66.0 ± 5.7^{a} | 65.6 ± 6.6^{a} | 126.5 ± 3.8^{bc} |
| Choles- | | | | | | | | | |
| terol | | | | | | | | | |
| (mg/ | | | | | | | | | |
| dL) | | | | | | | | | |
| LDL | 12.7 ± 1.1 | 52.4 ± 3.5 | 18.4 ± 2.3 | 15.3 ± 1.1 | 11.4 ± 1.2 | 12.8 ± 0.7 | <7 | <7 | 40.8 ± 3.0 |
| Choles- | | | | | | | | | |
| terol | | | | | | | | | |
| (mg/ | | | | | | | | | |
| dL) | | | | | | | | | |
| Triglyc- | 85.4 ± 5.3ª | 116.6 ± 6.2^{b} | 95.0 ± 5.0 [symbol] | 94.7 ± 9.5^{cdf} | 90.3 ± 5.7^{ad} | 83.9 ± 4.0^{af} | 80.6 ± 4.1^{e} | 70.4 ± 3.7^{e} | 112.1 ± 8.3^{b} [symbol] |
| erides | | | | | | | | | |
| (mg/ | | | | | | | | | |
| dL) | | | | | | | | | |

*Data are shown as means ± SE of 8-10 animals/group. A Tukey-Kramer post-hoc test was completed for significant difference comparison. Groups not sharing a common letter are significantly different. [symbol]
indicates data missing or illegible when filed

Total liver lipids were extracted using the method of Folch (Folch, 1; Lees, M.; Stanley, G. H. S. A SIMPLE METHOD FOR THE ISOLATION AND PURIFICATION OF TOTAL LIPIDES FROM ANIMAL TISSUES. Journal of Biological Chemistry 1957, 226(1), 497-509). Briefly, liver samples were thawed on ice and were homogenized with 2:1 chloroform-methanol mixture (v/v) to a final dilution of 20-fold the volume of the tissue sample. After homogenization, the crude extract is mixed thoroughly with 0.2 volume of PBS and vortex to mix thoroughly. The mixture is allowed to separate into two phases by centrifuging at 4000 r.p.m. for 15 min at 4° C. The upper phase is removed as much as possible with a pipette. The organic (lower) phase was air-dried in a fresh tube and resuspended in 200 μL of 1% Triton X-100 in ethanol. The suspension solutions were air dried and resuspended in PBS for the final lipid extract. Liver TC and TG were analyzed for cholesterol and triglycerides by using commercial kits (Abcam, Cambridge, MA).

Hepatic collagen content was measured via a hydroxyproline-based colorimetric assay using the sensitive total collagen assay (Quickzyme, Leiden, The Netherlands).

As shown in Tables 2 and 3, gel-encapsulated CGS26214 exhibited values closer to LC control, as compared to unencapsulated drug, which was much less effective in reversing or preventing the changes in biochemical markers induced by the high-fat diet.

### Example 6. Histology Analysis

Sections of fresh livers (not exceeding 0.5 cm in one dimension) from the left lateral lobes were fixed in 4% paraformaldehyde for 48-72 h, then stored in 75% (vol/vol) ethanol (Sigma-Aldrich, St. Louis, MO) for embedded in paraffin and 30% (wt/vol) sucrose (Sigma-Aldrich, St. Louis, MO) for embedded in optimal cutting temperature (OCT) compound. The liver samples were subsequently embedded, sectioned and stained with hematoxylin and eosin (H&E), Masson-trichrome and Oil red O by iHisto (Salem, MA).

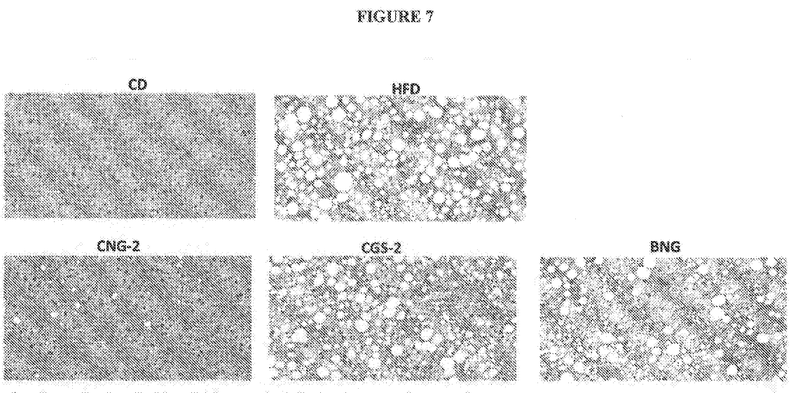

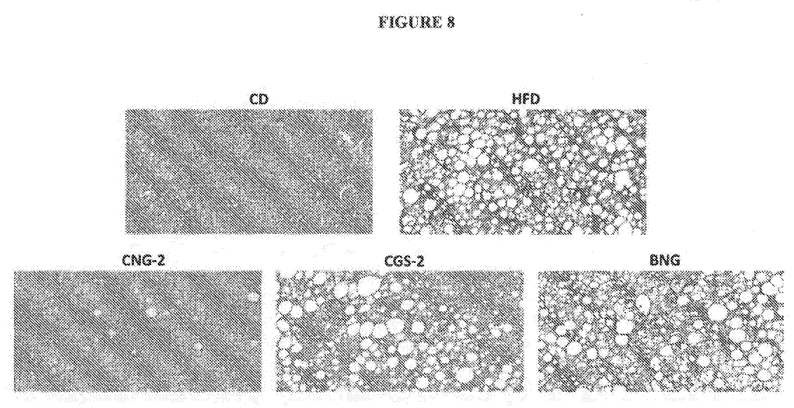

NASH was scored blindly by board-certified pathologists in H&E and Masson-trichrome stained cross-sections using an adapted version of scoring system for human NASH that developed by Kleiner et al. (Kleiner, D. E.; Brunt, E. M.; Van Natta, M.; Behling, C.; Contos, M. J.; Cummings, O. W.; Ferrell, L. D.; Liu, Y. C.; Torbenson, M. S.; Unalp-Adida, A.; et al. Design and Validation of a Histological Scoring System for Nonalcoholic Fatty Liver Disease. Hepatology 2005). Liver histology of mice from the 24 week study cohort is shown in FIG. 7 using hematoxylin-eosin staining to assess steatosis, inflammation and ballooning degeneration. As shown, mice from treatment groups HFD, CGS-2 and BNG show macrovesicular and microvesicular steatosis, inflammation, and ballooning degeneration, while mice from treatment groups CD and CNG-2 show no signs of macrovesicular and microvesicular steatosis, inflammation, and ballooning. FIG. 8 shows liver histology of mice from the 24 week study cohort using Masson's trichrome staining to assess fibrosis and collagen deposition. As shown, mice from treatment groups HFD, CGS-2 and BNG show signs of fibrosis and collagenous tissue fiber formation, while mice from treatment groups CD and CNG-2 show no signs of fibrosis or collagen deposition.

### Example 7. Quantitative RT-PCR Analysis

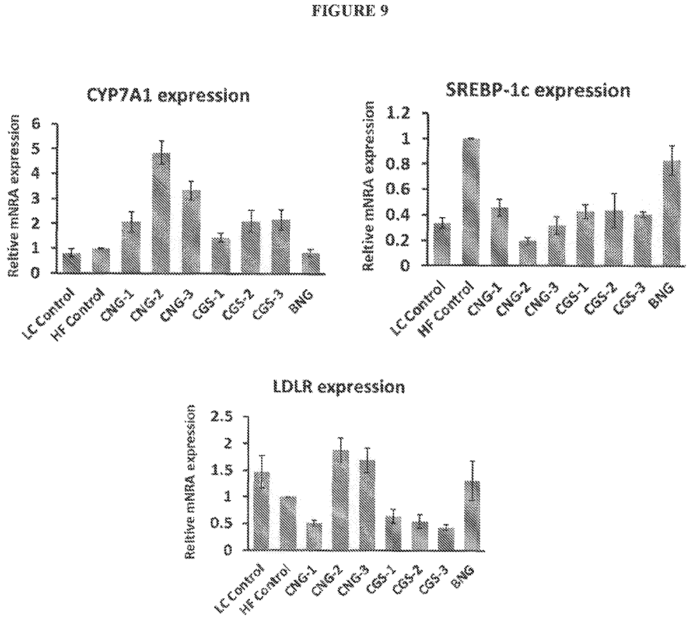

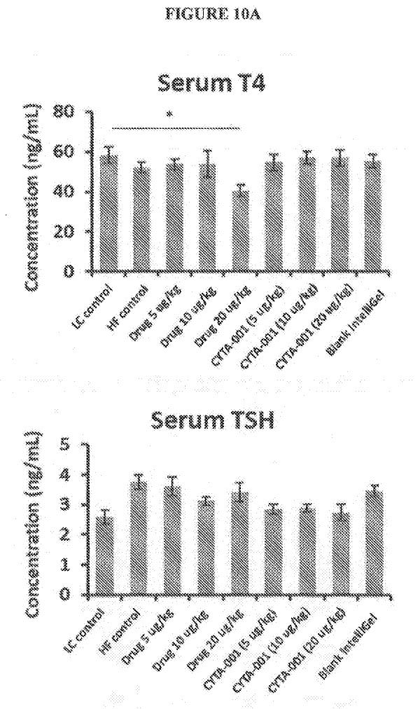

Sections of fresh livers (not exceeding 0.5 cm on one dimension) from the left lateral were collected and placed in 5-10 volumes of RNAlater® Solution (Invitrogen. Waltham, MA) for long-term storage. Total RNA was extracted from liver tissues using TRIzol reagent (Invitrogen, Carlsbad, CA). RNA integrity was examined by NanoDrop™ One/OneC Microvolume UV-Vis Spectrophotometer (Thermo Scientific, Waltham, MA). Contaminating gDNA was removed from total RNA samples using DNAse I (RNase-free)(New England Biolabs, Ipswich, MA). cDNA was synthesized from 2 μg of total RNA using High-Capacity cDNA Reverse Transcription Kit (Applied Biosystems, Foster City, CA). For quantitative PCR (qPCR), cDNA was amplified using PowerUp™ SYBR™ Green Master Mix (Applied Biosystems, Foster City, CA) and the StepOnePlus Real-Time PCR System (Applied Biosystems, Foster City, CA). The relative amount of each mRNA was calculated after normalization to the corresponding i-actin mRNA or GAPDH mRNA, and the ΔΔCt method was used for quantification. Assessment of gene transcription from liver tissue of mice cohorts from all treatment groups at both 12 weeks and 24 weeks was conducted. Gene expression of CYP7A1, SREBP-1c and LDLR were performed as all three genes are affected by activation of the TRβ receptor Results are shown in FIG. 9.

### Example 8. In Vivo T4 and TSH Analysis

A Diet Induced Obese (DIO) mouse model was used to study the in vivo effects of the CGS26214-encapsulated nanogels with anionic group-modified backbone (CGS-ANG), referred to as “CYTA-001,” on serum T4 and TSH within the context of obesity.

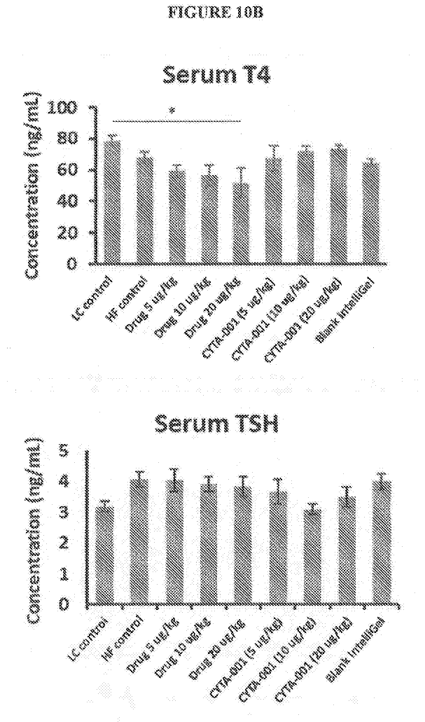

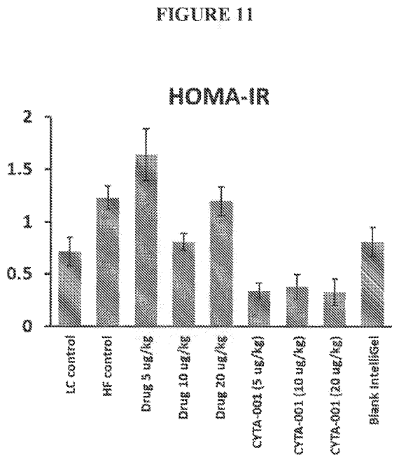

The DIO mice were treated with either the non-liver targeted TRβ agonist, Axitirome (CGS26214), also referred to as “drug” in FIGS. 10A and B, or CYTA-001 at 5 μg/kg, 10 μg/kg, or 20 μg/kg for 12 weeks or 24 weeks. Treated DIO mice serum T4/TSH levels were compared to a lean control (LC control), or low fat diet, and to a high fat control (HF control). After each designated treatment period, all the mice were sacrificed unfasted by cardiac puncture after gradual-fill CO₂ asphyxiation. Terminal blood samples were collected in SST-SERUM separator tubes for serum collection.

Serum was analyzed for thyroid hormone T4 and thyroid stimulating hormone (TSH) levels. The results, as shown in FIGS. 10A and 10B, indicated no statistically significant change in serum T4 or serum TSH levels in either the 12-week and 24-week timepoints for the thyromimetic-encapsulated gel (CYTA-001) compared to both the lean control and the high fat control, whereas the CGS26214-treated mice exhibited a statistically significant change in both serum T4 and TSH. These data suggested that CYTA-001 did not act peripherally and the observed effects on weight loss (e.g., Example 4) and on the metabolic parameters (e.g., Example 5) are indeed via the TRβ receptor in the liver. This is an unexpected result as thyromimetics have not been observed for weight loss without some level of peripheral activation of the TRα or TRβ receptor. Peripheral activation of the TRα receptor is generally toxic and ideally avoided.

### Example 9. In Vivo Insulin Resistance Analysis

DIO mice were used to study the in vivo effects of CYTA-001 on insulin resistance within the context of obesity and diet. Homeostatic model assessment for insulin resistance (HOMA-IR) levels were measured in DIO mice that were conditioned on high fat diets (HFD) for 24 weeks prior to a 5-week dosing regimen. The HFD DIO mice were treated with either the non-liver targeted TRβ agonist, Axitirome (CGS26214), also referred to as “drug” in FIG. 11, or CYTA-001 at 5 μg/kg, 10 μg/kg, or 20 μg/kg dosages for 5 weeks.

Results indicated, as shown in FIG. 11, that insulin resistance increases with the HFD and that insulin resistance normalizes upon treatment with CYTA-001 at all concentrations. The non-liver targeted CGS26214 was not observed to produce a significant effect on insulin resistance.

### Example 10. In Vivo Biodistribution Analysis

Male C57BL/6J mice were used to study the in vivo biodistribution of subcutaneously delivered empty nanogels with anionic group-modified backbone (BNG). Cy3@-tagged (fluorescent dye, ThermoFisher) BNG was subcutaneously injected and analyzed at various time points via W vivo fluorescence microscopy.

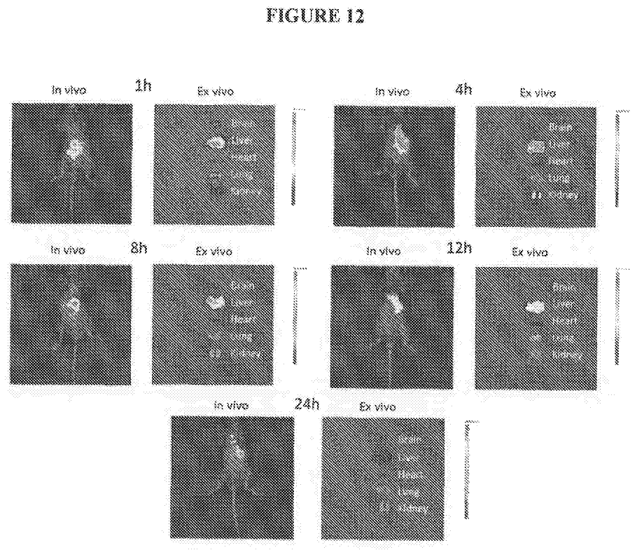

As shown in FIG. 12, fluorescence imaging indicated rapid targeting to and uptake in the liver within the first hour (h) after subcutaneous delivery. By 4 h post-delivery, the fluorescently-tagged BNG had begun being metabolized and excreted, as evidenced by the increasing intensity of fluorescence in the kidneys. Persistence in the liver was observed for up to 24 h post-injection. Imaging of the brain, heart, and lungs indicated little off-target accumulation.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compounds or compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Applicant's disclosure is described herein in preferred embodiments with reference to the figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### REFERENCES

1. WO 2012/162307 A2; U.S. Pat. No. 9,592,302 B2; U.S. Pat. No. 10,358,531 B2.
2. WO 2016044663 A2; U.S. Pat. No. 10,617,652 B2.
3. Reuben T. Chacko, Judy Ventura, Jiaming Zhuang, S. Thayumanavan, Polymer nanogels: A versatile nanoscopic drug delivery platform, Advance Drug Delivery Reviews, 64 (2012) 836-851.
4. Mallory R. Gordon, Jiaming Zhuang, Judy Ventura, Longyu Li, Kishore Raghupathi, and S. Thayumanavan, Biodistribution Analysis of NIR-Labeled Nanogels Using in Vivo FMT Imaging in Triple Negative Human Mammary Carcinoma Models, Mol. Pharmaceutics 2018, 15, 1180-1191 and Supporting Information, S1-S11.
5. Ja-Hyoung Ryu, Reuben T. Chacko, Siriporn Jiwpanich, Sean Bickerton, R. Prakash Babu, and S. Thayumanavan, Self-Cross-Linked Polymer Nanogels: A Versatile Nanoscopic Drug Delivery Platform, J. Am. Chem. Soc. 2010, 132, 17227-17235.
6. Tapan K. Majumdar, Shari Wu, Francis L. S. Tse, Quantitative determination of COS 26214, a cholesterol lowering agent, in human plasma using negative electrospray ionization liquid chromatography-tandem mass spectrometry, Journal of Chromatography B, 759 (2001) 99-108.
7. Zouhair F. Stephan, Edna C. Yurachek, Robin Sharif, Jong M. Wasvary, Kenneth S. Leonards, Chii-Whei Hu, Thomas H. Hintzeb, Ronald E. Steele, Demonstration of potent lipid-lowering activity by a thyromimetic agent devoid of cardiovascular and thermogenic effects, Atherosclerosis 126 (1996) 53 63.
8. Einav Yehuda-Shnaidman, et el., Thyroid Hormone, Thyromimetics, and Metabolic Efficiency, Endocrine Reviews, February 2014, 35(1):35-38.
9. Marta A. Kowalik, Amedeo Columbano and Andrea Perra, Thyroid Hormones, Thyromimetics and Their Metabolites in the Treatment of Liver Disease, Frontiers in Endocrinology, July 2018, Volume 9, Article 382.
10. Xuan G. Luong et al., Regulation of gene transcription by thyroid hormone receptor β agonists in clinical development for the treatment of non-alcoholic steatohepatitis (NASH), PLOS ONE, Dec. 11, 2020.
11. Amedeo Columbano, Grazia Chiellini and Marta Anna Kowalik, GC-1: A Thyromimetic With Multiple Therapeutic Applications in Liver Disease, Gene Expression, 2017, Vol. 17, pp. 265-275.
12. Rohit A. Sinha et al., Thyroid Hormones and Thyromimetics: A New Approach to Nonalcoholic Steatohepatitis?, Hepatology, VOL. 72, NO. 2, 2020.
13. Naim Alkhouri, Thyromimetics as emerging therapeutic agents for nonalcoholic steatohepatitis: rationale for the development of resmetirom (MGL-3196), Expert Opinion on Investigational Drugs 2020, VOL. 29, NO. 2, 99-101.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A pharmaceutical composition comprising a thyroid hormone receptor-β (TRβ) agonist or a TRβ antagonist encapsulated in a pharmaceutically acceptable particle carrier, wherein the TRβ agonist or TRβ antagonist is released upon degradation of the particle carrier.

2. The pharmaceutical composition of claim 1, wherein the particle carrier accumulates in or is targeted to an organ or tissue.

3. The pharmaceutical composition of claim 2, wherein the particle carrier is targeted to the liver.

4. The pharmaceutical composition of claim 3, wherein the particle carrier comprises a targeting agent.

5. The pharmaceutical composition of claim 4, wherein the targeting agent is an anionic functionality that targets organic anion-transporting polypeptide (OATP) group of receptors.

6. The pharmaceutical composition of claim 5, wherein the anionic functionality is mercaptopropionic acid.

7. The pharmaceutical composition of any one of claims 1 to 6, comprising a TRβ agonist.

8. The pharmaceutical composition of claim 7, wherein the TRβ agonist is axitirome (CGS26214).

9. The pharmaceutical composition of claim 7, wherein the TRβ agonist is Triiodothyronine (T3), Thyroxine (T4), Sobetirome (GC-1), Eprotirome (KB2115), Resmetirom (MGL-3196), VK2809, IS25, TG68, or CGS23425.

10. The pharmaceutical composition of any one of claims 1 to 9, wherein the particle carrier has an average diameter in the range of about 10 nm to about 200 nm, or in the range of about 20 nm to about 100 nm.

11. The pharmaceutical composition of claim 10, wherein the particle carrier has a zeta potential in the range of about −5 mV to about −40 mV.

12. The pharmaceutical composition of any one of claim 10 or 11, wherein the TRβ agonist or TRβ antagonist is incorporated in the particle carrier non-covalently.

13. The pharmaceutical composition of any one of claims 1 to 12, wherein the particle carrier is polymeric and comprises a crosslinked interior.

14. The pharmaceutical composition of claim 12 or 13, wherein degradation of the carrier is triggered by an increased concentration of a biochemical reductant.

15. The pharmaceutical composition of claim 14, wherein the particle carrier degrades in the presence of intracellular concentrations of glutathione (GSH).

16. The pharmaceutical composition of claim 15, wherein the particle carrier comprises an oligoethylene glycol (OEG) hydrophilic shell and a hydrophobic interior comprising disulfide-crosslinked branch groups.

17. The pharmaceutical composition of claim 16, wherein the particle carrier is formed by self-assembly in an aqueous environment.

18. The pharmaceutical composition of claim 17, wherein the particle carrier is formed in the presence of the TRβ agonist or TRβ antagonist and an amphiphilic copolymer, and wherein the amphiphilic copolymer comprises hydrophilic OEG branch groups and disulfide-linked hydrophobic branch groups to drive micellar assembly and agonist encapsulation, followed by cross-linking of hydrophobic branch groups through disulfide exchange reactions.

19. The pharmaceutical composition of claim 18, wherein the TRβ agonist or TRβ antagonist is not substantially released at concentrations of reducing agent found in normal blood plasma.

20. The pharmaceutical composition of any one of claims 15 to 19, wherein the OEG branch groups have from 5 to 50 ethylene glycol units.

21. The pharmaceutical composition of any one of claims 18 to 20, wherein the hydrophobic branch groups comprise pyridyldisulfide (PDS) moieties.

22. The pharmaceutical composition of any one of claims 18 to 21, wherein the OEG branch groups and the hydrophobic branch groups are present at a ratio of from about 1:4 to about 4:1.

23. The pharmaceutical composition of any one of claims 18 to 22, wherein the amphiphilic co-polymer is prepared by reversible addition fragmentation chain transfer (RAFT) polymerization of pyridyl disulfide ethyl methacrylate (PDSEMA) and oligoethylene glycol monomethyl ether methacrylate.

24. The pharmaceutical composition of claim 23, wherein the disulfide exchange reaction shuffles sulfhydryl groups of dithiothreitol (DTT) into the disulfides of disulfide-linked hydrophobic branch groups.

25. The pharmaceutical composition of claim 24, wherein the particle carrier has a crosslinking density from about 10% to about 70%, or from about 20% to about 60%, or from about 30% to about 50% relative to the total number of disulfide-containing structural units in the polymer.

26. The pharmaceutical composition of any one of claims 1 to 25, wherein the pharmaceutical composition is formulated for parenteral or enteral administration.

27. A pharmaceutical composition comprising a particle carrier non-covalently encapsulating an effective amount of a TRβ agonist; wherein:
the particle carrier comprises an anionic functionality that targets organic anion-transporting polypeptide (OATP) group of receptors, and the particle carrier comprises a disulfide-crosslinked polymeric interior that is not substantially degraded in normal blood plasma and is substantially degraded in the presence of intracellular concentrations of glutathione (GSH).

28. The pharmaceutical composition of claim 27, wherein the anionic functionality is mercaptopropionic acid.

29. The pharmaceutical composition of claim 27 or 28, wherein the TRβ agonist is axitirome (CGS26214).

30. The pharmaceutical composition of any one of claims 27 to 29, wherein the particle carrier has an average diameter in the range of about 10 nm to about 200 nm, or in the range of about 20 nm to about 100 nm.

31. The pharmaceutical composition of claim 30, wherein the particle carrier has a zeta potential in the range of about −5 mV to about −40 mV.

32. The pharmaceutical composition of any one of claims 27 to 31, wherein the particle carrier comprises an oligoethylene glycol (OEG) hydrophilic shell.

33. The pharmaceutical composition of claim 32, wherein the particle carrier is formed by self-assembly in an aqueous environment.

34. The pharmaceutical composition of claim 33, wherein the particle carrier is formed in the presence of the TRβ agonist and an amphiphilic copolymer, and wherein the amphiphilic copolymer comprises hydrophilic OEG branch groups and disulfide-linked hydrophobic branch groups to drive micellar assembly and agonist encapsulation, followed by cross-linking of hydrophobic branch groups through disulfide exchange reactions.

35. The pharmaceutical composition of claim 34, wherein the disulfide exchange reaction shuffles sulfhydryl groups of dithiothreitol (DTT) into the disulfides of disulfide-linked hydrophobic branch groups.

36. The pharmaceutical composition of any one of claims 32 to 35, wherein the OEG branch groups have from 5 to 50 ethylene glycol units.

37. The pharmaceutical composition of any one of claims 34 to 36, wherein the hydrophobic branch groups comprise pyridyldisulfide (PDS) moieties.

38. The pharmaceutical composition of any one of claims 34 to 37, wherein the OEG branch groups and the hydrophobic branch groups are present at a ratio of from about 1:4 to about 4:1.

39. The pharmaceutical composition of any one of claims 33 to 38, wherein the amphiphilic co-polymer is prepared by reversible addition fragmentation chain transfer (RAFT) polymerization of pyridyl disulfide ethyl methacrylate (PDSEMA) and oligoethylene glycol monomethyl ether methacrylate.

40. The pharmaceutical composition of claim 39, wherein the particle carrier has a crosslinking density from about 10% to about 70%, or from about 20% to about 60%, or from about 30% to about 50% relative to the total number of disulfide-containing structural units in the polymer.

41. A method for treating a disease or condition, comprising administering an effective amount of the pharmaceutical composition of any one of claims 1 to 40 to a patient in need thereof.

42. The method of claim 41, wherein the pharmaceutical composition is administered by intravenous or intraarterial administration, oral administration, or subcutaneous administration.

43. The method of claim 41 or 42, wherein the pharmaceutical composition is administered from once daily to about once monthly.

44. The method of any one of claims 41 to 43, wherein the patient has non-alcoholic fatty liver disease (NAFLD), non-alcoholic steatohepatitis (NASH), or a related disease or condition, and the particle carrier encapsulates a TRβ agonist.

45. The method of any one of claims 41 to 43, wherein the patient is a liver transplant recipient, a liver transplant donor, or the patient has cirrhotic liver disease, alcoholic liver disease, liver fibrosis, or acute liver failure, or a related disease or condition.

46. The method of any one of claims 41 to 43, wherein the patient has type 1 or type 2 diabetes or metabolic syndrome, or a related disease or condition.

47. The method of any one of claims 41 to 46, wherein the patient has elevated cholesterol, elevated triglycerides, or hyperlipidemia, or a related disease or condition.

48. The method of any one of claims 41 to 47, wherein the patient has insulin resistance.

49. The method of claim 41 or 48, wherein the patient is obese or overweight, or a related disease or condition.

50. A method for treating a condition selected from non-alcoholic fatty liver disease (NAFLD), non-alcoholic steatohepatitis (NASH), hypercholesterolemia, hyperlipidemia, metabolic syndrome, and obesity, or a related disease or condition, the method comprising:
administering an effective amount of a pharmaceutical composition comprising a particle carrier non-covalently encapsulating a TRβ agonist; wherein:
the particle carrier comprises an anionic functionality that targets organic anion-transporting polypeptide (OATP) group of receptors, and the particle carrier comprises a disulfide-crosslinked polymeric interior that is not substantially degraded in normal blood plasma and is substantially degraded in the presence of intracellular concentrations of glutathione (GSH).

51. The method of claim 50, wherein the anionic functionality is derived from mercaptopropionic acid.

52. The method of claim 50 or 51, wherein the TRβ agonist is axitirome (CGS26214).

53. The method of any one of claims 50 to 52, wherein the particle carrier has an average diameter in the range of about 10 nm to about 200 nm, or in the range of about 20 nm to about 100 nm.

54. The method of claim 53, wherein the particle carrier has a zeta potential in the range of about −5 mV to about −40 mV.

55. The method of any one of claims 50 to 54, wherein the particle carrier comprises an oligoethylene glycol (OEG) hydrophilic shell.

56. The method of claim 55, wherein the particle carrier is formed by self-assembly in an aqueous environment.

57. The method of claim 56, wherein the particle carrier is formed in the presence of the TRβ agonist and an amphiphilic copolymer, and wherein the amphiphilic copolymer comprises hydrophilic OEG branch groups and disulfide-linked hydrophobic branch groups to drive micellar assembly and agonist encapsulation, followed by cross-linking of hydrophobic branch groups through disulfide exchange reactions.

58. The method of claim 57, wherein the disulfide exchange reaction shuffles sulfhydryl groups of dithiothreitol (DTT) into the disulfides of disulfide-linked hydrophobic branch groups.

59. The method of any one of claims 55 to 58, wherein the OEG branch groups have from 5 to 50 ethylene glycol units.

60. The method of any one of claims 57 to 59, wherein the hydrophobic branch groups comprise pyridyldisulfide (PDS) moieties.

61. The method of any one of claims 57 to 60, wherein the OEG branch groups and the hydrophobic branch groups are present at a ratio of from about 1:4 to about 4:1.

62. The method of any one of claims 57 to 61, wherein the amphiphilic co-polymer is prepared by reversible addition fragmentation chain transfer (RAFT) polymerization of pyridyl disulfide ethyl methacrylate (PDSEMA) and oligoethylene glycol monomethyl ether methacrylate.

63. The method of any one of claims 57 to 62, wherein the particle carrier has a crosslinking density from about 10% to about 70%, or from about 20% to about 60%, or from about 30% to about 50% with respect to the total number of disulfide-containing structural units in the polymer.

64. A nanogel comprising a crosslinked copolymer and a thyroid hormone receptor-n (TRβ) agonist or a TRβ antagonist encapsulated in the crosslinked polymer.

65. The nanogel of claim 64, wherein the crosslinked copolymer comprises structural units of: 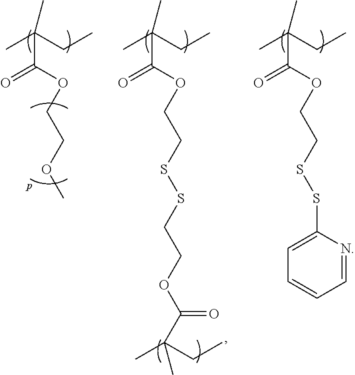
and

66. The nanogel of claim 64 or 65, wherein the crosslinked copolymer comprises the structural formula: 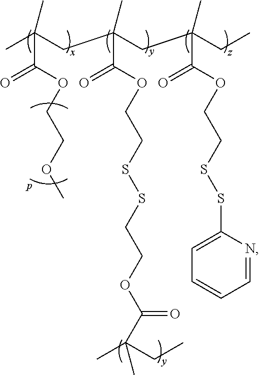
wherein each of x, y and z is independently a positive integer in the range from 1 to about 100.

67. The nanogel of any one of claims 64-66, wherein the crosslinked copolymer further comprises a targeting moiety adapted to accumulate in a target tissue or organ.

68. The nanogel of claim 67, wherein the target tissue or organ is liver.

69. The nanogel of claim 67 or 68, wherein the targeting moiety comprises a carboxylate.

70. The nanogel of claim any one of claims 67-69, wherein the targeting moiety comprises the structural unit of:

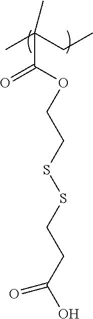

71. The nanogel of claim 70, wherein the crosslinked copolymer comprises the structural formula: 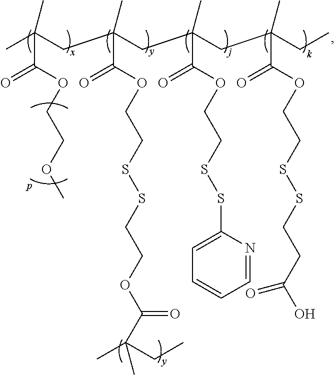
wherein j is a percentage of (x+y+j+k) in the range from 0% to about 70%, x and k are independently in the range from 1% to about 50%.
in the range from 0% to about 70%, and k is in the range from 1% to about 50%.

72. The nanogel of any one of claims 64-71, comprising a TRβ agonist.

73. The nanogel of any one of claims 64-73, comprising a TRβ antagonist.

74. The nanogel of any one of claims 64-73, wherein the TRβ agonist or TRβ antagonist is axitirome (CGS26214), Triiodothyronine (T3), Thyroxine (T4), Sobetirome (GC-1), Eprotirome (KB2115), Resmetirom (MGL-3196), VK2809, 1S25, TG68, or CGS23425.

75. The nanogel of any one of claims 64-74, wherein the crosslinked copolymer is characterized by a crosslinking density in the range from about 10% to about 70%.

76. The nanogel of any one of claims 64-75 in the form of nanoparticles having an average diameter in the range from about 10 nm to about 200 nm.

77. A pharmaceutical composition comprising the nanogel of any one of claims 64-76.

78. The pharmaceutical composition of claim 77 further comprising a pharmaceutically acceptable excipient, carrier, or diluent.

79. A method for treating a disease or condition, comprising administering to a patient in need thereof an effective amount of the pharmaceutical composition of claim 77 or 78.

80. The method of claim 79, wherein the disease or condition is selected from the group consisting of non-alcoholic fatty liver disease (NAFLD), non-alcoholic steatohepatitis (NASH), hypercholesterolemia, hyperlipidemia, metabolic syndrome, and obesity, or a related disease or condition.
